(19) United States
(12) Patent Application Publication
(10) Pub. No.: US 2006/0147449 A1
(43) Pub. Date: Jul. 6, 2006

(54) Method of using CD100 (or Sema4D) to mediate platelet activation and inflammatory responses

(76) Inventors: Lawrence F. Brass, Bala Cynwyd, PA (US); Li Zhu, Voorhees, NJ (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/280,785

(22) Filed: Nov. 15, 2005

Related U.S. Application Data
(60) Provisional application No. 60/627,825, filed on Nov. 15, 2004.

(51) Int. Cl. A61K 39/395; A61K 38/46; C12P 21/06

(52) U.S. Cl. 424/143.1; 424/94.65; 435/69.1

(57) ABSTRACT

This invention relates to methods of treating platelet disorders and/or endothelial cell disorders by modulating sema4D/CD100 activity. Specifically, this invention involves the use of compounds to increase or decrease the level of soluble sema4D/CD100.

### REFERENCE TO RELATED APPLICATION

This application claims priority to U.S. Provisional Application No.: 60/627,825, filed Nov. 15, 2004, the content of which is herein incorporated by reference.

### GOVERNMENT INTEREST

This invention was supported in part by funds from the U.S. Government (National Institutes of Health Grant No. P50-HL81012) and the U.S. Government may therefore have certain rights in the invention.

### FIELD OF THE INVENTION

The invention relates to the role of sema4D/CD100 in platelet activation. More particularly, this invention relates to methods of treating platelet disorders and/or endothelial cell disorders by inhibiting or activating sema4D/CD100.

### BACKGROUND

Platelet activation is classically defined in terms of the events that initiate the process, for example, the exposure of collagen within the vessel wall or the local accumulation of thrombin. When platelets come into close proximity with each other and with other cell types, they develop contacts only after platelet aggregation has begun. This contact provides the opportunity to have molecules on the surface of one platelet interact directly with molecules on the surface of another platelet (Brass, L. F., et al., Semin Thromb Hemost, 2004. 4: p. 399-410). Examples of such molecules include cell adhesion molecules, such as αIIbβ3 and JAM-1, and receptor:ligand pairs, such as Eph kinases and ephrins.

Electron micrographs show that the gap between aggregated platelets is 10-50 nm and that many of the platelets within an aggregate retain their secretory granules (Humbert, M., et al., Arterioscler Thromb Vasc Biol, 1996. 12: p. 1532-43). Discharge of the granule contents into the gap between platelets or between platelets and other nearby cells would allow the accumulation of the granule contents in a protected environment where higher local concentrations can be achieved. The rapid accumulation of platelets in a growing thrombus means that bioactive molecules that are secreted or shed by activated platelets can reach a higher local effective concentration. When flow is obstructed, such molecules would have an even greater ability to accumulate, potentially affecting not only other platelets, but also endothelial cells, vascular smooth cells and the array of monocytes, neutrophils and lymphocytes that traverse the area of injury and become involved in the host response. There is evidence to support such a mechanism including the deposition of PF4 from platelet α-granules in atherosclerotic plaques (Pitsilos, S., et al., Thromb Haemost, 2003. 6: p. 1112-20.; Sachais, B. S., et al., Semin Thromb Hemost, 2004. 3: p. 351-8) and the correlations that are being found between CD40 ligand levels and the presence of significant cardiovascular disease (Anand, S. X., et al., Thromb Haemost, 2003. 3: p. 377-84). For example, CD40L is a protein that is recruited to the surface of activated platelets and then cleaved, releasing a bioactive ectodomain fragment (Prasad, K. S., et al., Curr Opin Hematol, 2003. 5: p. 356-61).

Sema4D/CD100 is similar to CD40L, but is a member of the semaphorin family of proteins. Semaphorins are a large family of signaling molecules with at least twenty members that have been divided into seven classes defined in part on whether or not they are secreted, held to the cell surface by a GPI anchor or have an integral transmembrane domain (Tamagnone, L. and P. M. Comoglio, Trends Cell Biol, 2000. 9: p. 377-83; Yu, H. H. and A. L. Kolodkin, Neuron, 1999. 1: p. 11-4). Structurally, the semaphorins have a common 500 amino acid extracellular “sema” domain that forms a 7-bladed propeller structure (Love, C. A., et al., Nat Struct Biol, 2003. 10: p. 843-8). The remainder of the semaphorin sequence and structure is much more variable. Semaphorins are best known for their role in neuronal guidance, where they typically trigger retraction, but semaphorins also play a role in immune regulation (Kumanogoh, A. et. al. Trends Immunol, 2001. 12: p. 670-6) and a receptor for sema4D/CD100 was recently identified in endothelial cells (Basile, J. R., et al., Cancer Res, 2004. 15: p. 5212-24).

Sema4D/CD100 is a 150 kDa type I membrane glycoprotein with an extracellular N-terminus, followed by a sema domain, an immunoglobulin (Ig)-like domain, a lysine-rich domain, a transmembrane domain and a 110 residue cytoplasmic tail with consensus tyrosine and serine phosphorylation sites (Hall, K. T., et al., Proc Natl Acad Sci USA, 1996. 21: p. 11780-5; Furuyama, T., et al., J Biol Chem, 1996. 52: p. 33376-81). Sema4D/CD100 is widely distributed in the central nervous system, but can also be found in thymus, spleen and kidney, and expression has been reported in lymphocytes, monocytes, NK cells and neutrophils (Hall, K. T., et al., Proc Natl Acad Sci USA, 1996. 21: p. 11780-5; Furuyama, T., et al., J Biol Chem, 1996. 52: p. 33376-81; Bougeret, C., et al., J Immunol, 1992. 2: p. 318-23; Moreau-Fauvarque, C., et al., J Neurosci, 2003. 27: p. 9229-39. Granziero, L., et al., Blood, 2003. 101: p. 1962-1969; Giraudon, P., et al., J Immunol, 2004. 2: p. 1246-1255). On nonreducing gels, sema4D/CD100 runs as a 300 kDa homodimer, as well as a 150 kDa monomer. Dimerization requires a cysteine residue (Cys674) located in the extracellular domain near the transmembrane domain. The dimer is thought to be the active form (Delaire, S., et al., J Immunol, 2001. 7: p. 4348-54).

Sema4D/CD100 can undergo regulated shedding from the surface of cells that express it (Elhabazi, A., et al., J Immunol, 2001.7: p. 4341-7.; Wang, X., et al., Blood, 2001. 11: p. 3498-504). The cleavage site on sema4D/CD100 has not been identified, but since proteolysis of sema4D/CD100 occurs in lymphocytes (21) and platelets produces an ˜120 kDa fragment that runs as a dimer under nonreducing conditions, it is likely that the cleavage site is located in the short region between Cys674 and the transmembrane domain. Soluble sema4D/CD100 (sCD100) that is released from activated lymphocytes inhibits immune cell migration, acts as a co-stimulator for CD40-induced B-cell proliferation and immunoglobulin production, and induces release of pro-inflammatory cytokines by monocytes (Delaire, S., et al., J Immunol, 2001. 7: p. 4348-54; Wang, X., et al., Blood, 2001. 11: p. 3498-504; Kumanogoh, A., et al., Immunity, 2000. 5: p. 621-31. Ishida, I., et al., Int Immunol, 2003. 8: p. 1027-34). It also causes axonal repulsion or collapse (Moreau-Fauvarque, C., et al., J Neurosci, 2003. 27: p. 9229-39; Giraudon, P., et al., J Immunol, 2004. 2: p. 1246-1255). These responses are currently attributed to the ability of sema4D/CD100 to act as a ligand for two receptors: CD72 (Kumanogoh, A., et al., Immunity, 2000. 5: p. 621-31) and a higher affinity receptor called plexin-B1 (Kumanogoh, A., et al., Immunity, 2000. 5: p. 621-31; Tamagnone, L., et al., Cell, 1999. 1: p. 71-80). Sema4D/CD100 may play a role in signal transduction as anti-sema4D/CD100 antibodies cause increased T-cell proliferation if added in the presence of an anti-CD3 antibody and there are reports that sema4D/CD100 can associate with the tyrosine phosphatase, CD45, and an unknown serine kinase (Elhabazi, A., et al., J Immunol, 2001. 7: p. 4341-7; Billard, C., et al., Blood, 2000. 3: p. 965-72; Herold, C., et al., J Immunol, 1996. 12: p. 5262-8.). Sema4D/CD100 knockout mice are viable, but have defects in B-cell development, presumably related to a failure to interact with CD72 (Shi, W., et al., Immunity, 2000. 5: p. 633-42; Pan, C., N. Baumgarth, and J. R. Parnes, Immunity, 1999. 4: p. 495-506).

One known receptor for sema4D/CD100 is CD72. CD72 or Lyb-2 is a 45 kDa type II membrane protein that is a member of the C-type lectin family and is normally present as a homodimer (Nakayama, E., I. von Hoegen, and J. R. Parnes, Proc Natl Acad Sci USA, 1989. 4: p. 1352-6). It has an extracellular C-terminus and two immunoreceptor tyrosine-based inhibitory motif (ITIM) domains within the cytoplasmic N-terminus: 5ITYADL10 and 37LTYENV42, (Wu, Y., et al., Curr Biol, 1998. 18: p. 1009-17; Adachi, T., et al., J Immunol, 1998. 10: p. 4662-5). The former motif is necessary for the association of CD72 with the tyrosine phosphatase, SHP-1. The latter motif binds Grb2 in vitro. At least four splice variants of CD72 have been described (Ying, H., et al., J Immunol, 1995. 6: p. 2743-52), which differ primarily in the extracellular domain as the cytoplasmic domain is conserved. CD72 is expressed on all B-cells (but not plasma cells) and, at lower densities, on some T-cells (Robinson, W. H., M. M. Landolfi, and J. R. Parnes, Immunogenetics, 1997. 3: p. 195-200). Anti-CD72 antibodies can cause B-cell proliferation (Subbarao, B., J. Morris, and A. R. Baluyut, Cell Immunol, 1988. 2: p. 329-42), and enhance B-cell responses to IL-1 and IL-4. CD72 knockout mice have been generated which show normal numbers of pre-B cells in the bone marrow, but a decrease in the number of mature B-cells in the CD72 knockout mice and a 20-30% decrease in total B-cells in the spleen, lymph nodes and Peyer's patches (Pan, C., N. Baumgarth, and J. R. Parnes, Immunity, 1999. 4: p. 495-506).

Crosslinking of the B cell receptor increases both the tyrosine phosphorylation of CD72 and the association of SHP-1 with CD72, (Wu, Y., et al., Curr Biol, 1998. 18: p. 1009-17; Adachi, T., et al., J Immunol, 1998. 10: p. 4662-5; Hokazono, Y., et al., J Immunol, 2003. 4: p. 1835-43). Since binding of SHP-1 to CD72 occurs via an SH2 domain in SHP-1, thereby exposing the SHP-1 catalytic site, a proposed model is that SHP-1 associated with CD72 is activated and held close to its membrane-associated substrates. In this model, CD72/SHP-1 is best viewed as a negative regulator, providing a block to signaling that is relieved when CD72 is ligated, causing SHP-1 to dissociate and resume its inactive conformation (Kumanogoh, A. and H. Kikutani, Trends Immunol, 2001. 12: p. 670-6; Ishida, I., et al., Int Immunol, 2003. 8: p. 1027-34; Parnes, J. R. and C. Pan, Immunol Rev, 2000. 176: p. 75-85). The best known ligand for CD72 is sema4D/CD100, which causes the dissociation of SHP-1 from CD72 (Ishida, I., et al., Int Immunol, 2003. 8: p. 1027-34; Wu, Y., et al., Curr Biol, 1998. 18: p. 1009-17).

A second known receptor for sema4D/CD100 is a member of the plexin famly of proteins. Plexins are cell surface molecules best known as receptors for semaphorins, (Pasterkamp, R. J. and A. L. Kolodkin, Curr. Opin. Neurobiol., 2003. 13: p. 79-89; Tamagnone, L. and P. M. Comoglio, EMBO Rep, 2004.4: p. 356-61). There are 9 human genes encoding plexins, divided into four families. Plexins typically have an extracellular N-terminus with a Sema domain, a transmembrane domain and a conserved intracellular domain. Most are thought to form homodimers or multimers. Although plexins are best known for their role as repulsive axonal guidance molecules, plexin-B1 has been shown to be a receptor for sema4D/CD100, which it binds with nM affinity (Tamagnone, L., et al., Plexins are a large family of receptors for transmembrane, secreted, and GPI-anchored semaphorins in vertebrates. Cell, 1999. 1: p. 71-80). Unlike CD72, plexin-B1 is not prominently expressed on T- or B-cells, but it may be upregulated during T-cell activation (Granziero, L., et al., Blood, 2003. 101: p. 1962-1969; Ishida, I., et al., Int Immunol, 2003. 8: p. 1027-34). Based on Northern blots, plexin-B 1 is expressed in brain and kidney and it has recently been shown to be present in endothelial cells (Basile, J. R., et al., Cancer Res, 2004. 15: p. 5212-24). It is proposed that the ability of soluble sema4D/CD100 to inhibit monocyte migration is mediated by plexin-B1 since monocytes appear not to express CD72 (Delaire, S., et al., J Immunol, 2001. 7: p. 4348-54). Plexin-B1 appears to have several different mechanisms for transmitting signals which may be mediated by its C-terminal PDZ target domain (Aurandt, J., et al., Proc Natl Acad Sci USA, 2002. 19: p. 12085-90; Perrot, V., J. Vazquez-Prado, and J. S. Gutkind, J Biol Chem, 2002. 45: p. 43115-20; Swiercz, J. M., et al., Neuron, 2002. 1: p. 51-63; Oinuma, I., et al., J Biol Chem, 2003. 28: p. 25671-7; Oinuma, I., et al., Science, 2004. 5685: p. 862-5).

To date, prior to the present invention, there was no evidence to show that sema4D/CD100 is capable of regulating platelet activity. Given that such a determination would be useful in potential therapeutics involving both platelet disorders, endothelial disorders and wound healing, there is an ongoing need in the art for identifying molecules that regulate this pathway.

### SUMMARY

In one aspect, the invention provides a method of treating at least one symptom of a platelet disorder or an endothelial cell disorder in a human, said method comprising administering a sema4D/CD100 modulator to said human.

In another aspect, the invention provides a method for identifying a test compound which modulates sema4D/CD100 activity, said method comprising: (a) contacting a cell expressing sema4D/CD100 with said test compound; (b) measuring an activity of sema4D/CD100 in the presence of the test compound and in the absence of the test compound; and (c) comparing said activity of sema4D/CD100 in the presence of said test compound with said activity of sema4D/CD100 in the absence of said test compound, thereby determining sema4D/CD100 activity of the test compound.

In another aspect, the invention provides a method of screening for a compound having anti-thrombotic or anti-platelet activity comprising: (a) contacting platelets in vitro with a test compound; and (b) monitoring activity of said compound to inhibit platelet aggregation through inhibition of sema4D/CD100 cleavage, wherein activity as an inhibitor of sema4D/CD100 binding is indicative of anti-thrombotic or anti-platelet activity of said compound.

In another aspect, the invention provides a method of diagnosing a platelet disorder and/or endothelial cell disorder or similar condition in a mammal, said method comprising: (a) obtaining a biological sample from said mammal; (b) determining a presence or level of sema4D/CD100 in said biological sample; (c) comparing the level of sema4D/CD100 in said biological sample with the level of sema4D/CD100 in a biological sample obtained from a like mammal not afflicted with a platelet disorder and/or endothelial cell disorder, wherein a higher level of sema4D/CD100 in said biological sample from said mammal compared with the level of sema4D/CD100 in said biological sample from said like mammal is an indication that said mammal is afflicted with a platelet disorder and/or endothelial cell disorder, thereby diagnosing the platelet disorder and/or endothelial cell disorder in said previously undiagnosed mammal.

In another aspect the invention provides kits for performing the methods of the invention.

Additional objects, advantages and novel features of the invention will be set forth in part in the description, examples and figures which follow, and in part will become apparent to those skilled in the art on examination of the following, or may be learned by practice of the invention.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1A depicts the expression of sema4D/CD100 in human t(A) lysates from platelets, SK-N-SH neuroblastoma cells and human brain were immunoblotted with an antibody (Abm30) directed against the sema4D C-terminus. FIG. 1B represents human platelet lysate was subjected to SDS-PAGE under reducing or non-reducing conditions as indicated, and then immunoblotted with Abm30. FIG. 1C represents gel-filtered platelets incubated with or without PMA for 15 minutes in the absence of stirring were stained with either a FITC-conjugated antibody (A8) directed against the N-terminus of sema4D or FITC-conjugated isotype-matched mouse IgG and then analyzed by flow cytometry.

FIG. 2 shows that platelet aggregation induces sema4D/CD100 cleavage. FIG. 2A represents PMA-treated platelets were stained for sema4D and analyzed by flow cytometry. The results are expressed relative to the amount of sema4D present on resting platelets (mean±SEM, N=3). FIG. 2B represents washed platelets that were stimulated with collagen (50 μg/ml) and allowed to aggregate at 37° C. Lysates were immunoblotted with the C-terminal sema4D antibody, Abm30. FIG. 2C represents gel-filtered platelets were incubated with thrombin (0.5 u/ml) or PMA (100 nM) at 37° C. without stirring. Sema4D was precipitated and blotted with Abm30. FIG. 2D represents platelets were surface-labeled with biotin-7-NHS and incubated with SFLLRN (10 μM) for 15 min without stirring. (left) After centrifugation, sema4D in the fluid phase was precipitated with BB18 and detected with an anti-biotin antibody. (right) Platelet lysates were precipitated with anti-biotin and blotted with Abm30.

FIG. 3 shows that platelet aggregation enhances agonist-induced sema4D cleavage. Washed platelets were stimulated with 100 nM PMA in an aggregometer cuvette at 37° C. with or without stirring. Lysates were precipitated and blotted with antibody Abm30. The extent of platelet aggregation in the stirred samples is indicated below each lane.

FIG. 4 represents the effect of metalloprotease inhibitors on sema4D expression. FIG. 4A shows platelets that were preincubated with 20 μM TAPI-2 (negative control) for 60 min, and then stimulated with 100 nM PMA at 37° C. for 30 min. Sema4D was precipitated and blotted with Abm30. Ig light and heavy chains are indicated. FIG. 4B shows platelet suspensions pretreated with or without TAPI-2 were incubated with PMA for 15 min or 60 min, stained for sema4D and analyzed by FACS (mean±SEM from 3 studies).

FIG. 5 shows ADAM17 is present in human platelets and is necessary for cleavage of sema4D. FIG. 5A represents an immunoblot of human platelet lysate using antibody Tc3-7.49 directed against the ADAM 17 catalytic domain, then re-probed with an antibody to the β subunit of αIIbβ3 as a loading control. The lysates were prepared from platelets incubated with or without 100 nM PMA for 30 minutes at 37° C. with or without stirring as noted. FIG. 5B represents an immunoblot of glycoproteins harvested by affinity chromatography from lysates of platelets and Cos-7 cells.

FIG. 6 represents collagen-induced platelet aggregation in control (WT) and sema4D/CD100 (−/−) mice. Duplicate runs from a single experiment are shown. The final collagen concentration was 3 μg/ml.

FIG. 7 represents an immunoblot for pAkt473 from human platelets that were stimulated with recombinant sema4D or thrombin. The results show that soluble sema4D/CD100 stimulates Akt phosphorylation in human platelets.

FIG. 8 represents collagen-induced Akt phosphorylation in platelets from control (WT) and sema4D/CD100 (−/−) mice. The lysates were prepared and blotted for pAkt473, then stripped and reprobed for total Akt as indicated.

FIG. 9 shows that soluble sema4D/CD100 potentiates platelet aggregation. FIG. 9A represents human platelets that were stimulated with a low concentration of ADP±soluble a soluble chimera of the extracellular domain of sema4D/CD100 fused to IgGK Fc. FIG. 9B represents an immunoblot of FcCD100 that was performed under reducing and non-reducing conditions using anti-Fc.

FIG. 10 represents the nucleic acid sequence where ADAM17/TACE may cleave sema4D/CD100 between the cysteine responsible for homodimerization and the transmembrane domain (underlined). Note that the human and mouse sequences are virtually identical in this region.

FIG. 11 represents the cleavage of sema4D by ADAM17. Recombinant ADAM17 was incubated with Fc-sema4D in which the extracellular domain of human sema4D was joined via a linker to the N-terminus of IgG1 Fc. Two fragments were detected by amido black protein staining (left), the smaller of which (fragment #1) was also detectable with anti-Fc, as was the intact protein (middle). The N-terminal sequence of fragment #1 was determined.

FIG. 12 represents CD72 expression in platelets. FIG. 12A represents immunoblots performed with two different CD72 antibodies. Results in platelets are compared with those obtained with B-cells and with HEK293 cells transfected with human CD72 or empty vector (middle). FIG. 12B represents a FACS analysis of resting and activated platelets with either FITCconjugated anti-CD72 or a FITC conjugated isotype-matched control antibody.

FIG. 13 depicts the association of SHP-1 with CD72 in platelets. FIG. 13 represents human platelets that were incubated with an agonist or with FcCD100 or Fc, then lysed and immunoprecipitated with rabbit antiCD72 (or rabbit IgG) before being immunoblotted with monoclonal anti-SHP-1 or anti-CD72 as indicated.

### DESCRIPTION OF PREFERRED EMBODIMENTS OF THE INVENTION

The present invention provides novel methods of treating platelet and/or endothelial cell disorders by modulating the activity of sema4D/CD100. This invention shows that platelet activation causes additional sema4D/CD100 to be recruited to the platelet surface, after which its exodomain is cleaved and shed by a metalloprotease, for example the TNF-α cleaving enzyme, TACE which is also know as ADAM17.

In one aspect the invention provides a method of treating at least one symptom of a platelet disorder and/or endothelial cell disorder in a mammal, said method comprising administering a sema4D/CD100 inhibitor to said mammal.

As used herein, the phrase “soluble sema4D/CD100” refers to a fragment of sema4D/CD100 that is cleaved during platelet activation. In an embodiment, cleavage occurs by a metalloprotease. In a preferred embodiment, the metalloprotease is ADAM17 or TNF-α converting enzyme (TACE).

As used herein, the term “modulator” refers to any molecule that affects the activity, expression or amount of cleavage of sema4D/CD100. In an embodiment, the modulator increases the activity, expression or amount of cleavage of sema4D/CD100. In another embodiment, the modulator decreases the activity, expression or amount of cleavage of sema4D/CD100. In another embodiment, the term “modulator” refers to a molecule that prevents or inhibits binding of sema4D/CD100 to its receptor. In a preferred embodiment, the sema4D/CD100 receptor is expressed by a platelet, endothelial cell or monocyte. In another embodiment, the receptor is CD72 or plexin B 1. In an embodiment, the modulator at least one type of cell. For example, a modulator may affect sema4D/CD100 activity on a platelet alone or a platelet and an endothelial cell. In another embodiment, the modulator binds to sema4D/CD100 directly. In another embodiment, the modulator binds to a metalloprotease that cleaves sema4D/CD100. The modulator includes but is not limited to agonists, antagonists, antibodies and mimetics.

As used herein, the term “angiogenesis” refers to the process of growing at least one new blood vessel. In one embodiment, angiogenesis occurs in a cancer.

As used herein, the term “cleavage” refers to an enzymatic reduction in size of sema4D/CD100. In an embodiment, the cleavage occurs by a metalloprotease.

The skilled artisan will readily understand, when armed with the present disclosure and the data disclosed herein, that diagnostics can be developed which are capable of detecting the soluble sema4D/CD100 in a mammal. This is because, as demonstrated by the data elsewhere herein, occurs with the onset of platelet aggregation and thrombosis. Thereby, determining the level of soluble sema4D/CD100 in a mammal or cell can be used as a powerful and novel diagnostic technique for the detection of platelet disorders and diagnosis of patients with an increased tendency to acquire a platelet disorders and/or endothelial cell disorder, and the like. Thus, the present invention further encompasses methods for the diagnosis of platelet disorders and predicting a high potential to get either of these disorders. In all instances recited herein, whether treating or diagnosing platelet disorders or endothelial cell disorders, the most preferred subject to be treated is a mammal. In an embodiment, the mammal is a human.

In an embodiment, the platelet disorder is selected from the group consisting of heart attack, stroke, cardiopulmonary bypass disease, heparin-induced thrombocytopenia, atherosclerosis, thrombosis, thrombotic microangiopathy, disseminated intravascular coagulation, acquired platelet disorder and inherited platelet disorder. In another embodiment, the endothelial cell disorder is selected from the group consisting of arteriosclerosis, atherosclerosis, thrombosis and aneurysm.

In an embodiment, the present invention provides a method of diagnosing a platelet disorder or endothelial cell disorder in a mammal. The method comprises obtaining a biological sample from the mammal and then determining a presence or level sema4D/CD100 in said biological sample. The method then comprises comparing the level sema4D/CD100 in the biological sample with the level of sema4D/CD100 in a biological sample obtained from a like mammal not afflicted with a platelet disorder or endothelial cell disorder. In an embodiment, a higher level of sema4D/CD100 in the biological sample from the mammal compared with the level of sema4D/CD100 in the biological sample from the like mammal is an indication that the mammal is afflicted with a platelet disorder, thereby diagnosing platelet disorder or endothelial cell disorder in the previously undiagnosed mammal. In a preferred embodiment, the level of sema4D/CD100 that is detected is the level of soluble sema4D/CD100. In another preferred embodiment, the soluble sema4D/CD100 is the exodomain of sema4D/CD100.

One skilled in the art would understand, based upon the disclosure provided herein, that there are a wide variety of methods for assessing the level of sema4D/CD100 in a sample. Such methods include, but are not limited to, Real Time PCR, which can quantitatively measure mRNA expression, antibody-based detection methods (e.g., using anti-sema4D/CD100 antibodies in Western blot or other immune-based analyses, such as ELISA or enzyme linked immuno-sandwich assay); methods for assessing the level of sema4D/CD 100 expression in a cell and/or tissues (e.g., using polymerase chain reaction (PCR), and Northern blot analysis, and the like), and/or methods such as DNA/protein binding assays (i.e. EMSAs) based on detection of binding with a duplex nucleic acid, e.g., soluble sema4D/CD100 polypeptide. Thus, methods of detecting platelet disorders and/or endothelial disorders, either by detecting a sema4D/CD100 polypeptide or a nucleic acid encoding sema4D/CD100 (e.g., RNA or DNA) are disclosed herein or are well known to those skilled in the art and are encompassed in the present invention. Furthermore, the present invention encompasses similar assays for the detection of a specific protein or nucleic acid in a sample as may be developed in the future.

One of skill in the art will appreciate, when armed with the present disclosure and data herein, that methods for determining the level of sema4D/CD100 include, but are not limited to Western blotting, ELISA, and other immunodetection assays well known in the art.

In one aspect, the biological sample is selected from the group consisting of a blood sample or plasma sample and the like.

The invention provides a method of assessing the effectiveness of a treatment for platelet disorder and/or endothelial cell disorder by measuring the level of soluble sema4D/CD100. The method comprises assessing the level of sema4D/CD100, amount, and/or activity, before, during and after a specified course of treatment for a disease, disorder or condition mediated by or associated with increased sema4D/CD100 expression. This is because, as stated previously elsewhere herein, increased level of soluble sema4D/CD100, amount and/or activity is associated with or mediates a platelet disorder or endothelial cell disorder. In an embodiment, sema4D/CD100 activity is determined by measuring Akt phosphorylation.

Thus, assessing the effect of a course of treatment upon sema4D/CD100 expression/amount/activity indicates the efficacy of the treatment such that a lower level of soluble sema4D/CD100, a change in the amount of sema4D/CD100 or a change in sema4D/CD100 activity indicates that the treatment method was successful.

In an embodiment, the course of therapy to be assessed can include, but is not limited to anti-platelet agents or drugs.

Probes can be labeled by a variety of markers that are known in the art, including for example, radioactive markers, fluorescent markers, enzymatic markers, and chromogenic markers. The use of ³²P is but one example for marking or labeling a particular probe.

The invention further provides methods of isolating an sema4D/CD100 protein. As shown herein in the examples, sema4D/CD100 is associated with a platelet disorders and/or endothelial cell disorders. Thus, sema4D/CD100 protein can be isolated from a patient with platelet disorder and/or endothelial cell disorder by use of an antibody or a protein binding sequence. The biological sample used in the practice of this method can be any described herein. In an embodiment, the biological sample is a blood sample or plasma sample.

Thus, the invention further provides a method of detecting the presence of a polypeptide according to the invention in a sample, said method comprising the steps of bringing the sample to be tested into contact with an antibody directed against a polypeptide comprising an amino acid sequence of sema4D/CD100, a polypeptide fragment or variant of a polypeptide comprising an amino acid sequence of sema4D/CD100, and detecting the antigen/antibody complex formed.

Antibodies against sema4D/CD100 are made by methods known to the skilled artisan.

The generation of polyclonal antibodies is accomplished by inoculating the desired animal with the antigen and isolating antibodies which specifically bind the antigen therefrom.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Human monoclonal antibodies may be prepared by the method described in U.S. patent publication 2003/0224490. Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein. Further, the antibody of the invention may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention is also construed to include synthetic phage display libraries in which the heavy and light chain variable regions may be synthesized such that they include nearly all possible specificities (Barbas, 1995, Nature Medicine 1:837-839; de Kruif et al., 1995, J. Mol. Biol. 248:97-105).

A preferred method of co-immunoprecipitation is described in the examples herein. See also Harlow et al., (1988, Antibodies: A Laboratory Manual, Cold Spring Harbor Laboratory Press, Cold Spring Harbor, N.Y.) and Harlow et al., (1999, Using Antibodies: A Laboratory Manual, Cold Spring Harbor Laboratory Press, Cold Spring Harbor, N.Y.).

Various procedures known in the art may be used for the production of polyclonal antibodies to the sema4D/CD100 polypeptide or derivative or analog thereof. For the production of antibody, various host animals can be immunized by injection with the sema4D/CD100 polypeptide, or a derivatives (e.g., fragment or fusion protein) thereof, including but not limited to rabbits, mice, rats, sheep, goats, etc. In an embodiment, the sema4D/CD100 polypeptide or a fragment thereof can be conjugated to an immunogenic carrier, e.g. bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the sema4D/CD100 polypeptide, or a fragment, analog, or derivative thereof, any technique that provides for the production of antibody molecules by continuous cell lines in culture may be used. These include but are not limited to the hybridoma technique originally developed by Kohler and Milstein (1975, Nature, 256:495-497), as well as the trioma technique, the human B-cell hybridoma technique (Kozbor et al., 1983, Immunology Today, 4:72; Cote et al., 1983, Proc. Natl. Acad. Sci. U.S.A., 80:2026-2030), and the EBV-hybridoma technique to produce human monoclonal antibodies (Cole et al., 1985, In: Monoclonal Antibodies and Cancer Therapy, Alan R. Liss, Inc., pp. 77-96). In an additional embodiment of the invention, monoclonal antibodies can be produced in germ-free animals (WO 89/12690). In fact, according to the invention, techniques developed for the production of “chimeric antibodies” (Morrison et al., 1984, J. Bacteriol. 159:870; Neuberger et al., 1984, Nature, 312:604-608; Takeda et al., 1985, Nature 314:452-454) by splicing the genes from a mouse antibody molecule specific for the sema4D/CD100 polypeptide together with genes from a human antibody molecule of appropriate biological activity can be used; such antibodies are within the scope of this invention. Such human or humanized chimeric antibodies may be used in therapy of human diseases or disorders (described infra), since the human or humanized antibodies are much less likely than xenogenic antibodies to induce an immune response, in particular an allergic response, themselves.

According to the invention, techniques described for the production of single chain antibodies (U.S. Pat. Nos. 5,476,786 and 5,132,405 to Huston; U.S. Pat. No. 4,946,778) can be adapted to produce sema4D/CD100 polypeptide-specific single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for the sema4D/CD100 polypeptide, or its derivative, or analog.

Antibody fragments which contain the idiotype of the antibody molecule can be generated by known techniques. For example, such fragments include but are not limited to: the F(ab′) fragment which can be produced by pepsin digestion of the antibody molecule; the Fab′ fragments which can be generated by reducing the disulfide bridges of the F(ab′)2 fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays (see Methods in Molecular Biology, Vol. 149; The ELISA Guidebook by John R. Crowther, Humana Press, Totowa, N.J., 2001), immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In an embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of the sema4D/CD100 polypeptide, one may assay generated hybridomas for a product which binds to the sema4D/CD100 polypeptide fragment containing such epitope. For selection of an antibody specific to the sema4D/CD100 polypeptide from a particular species of animal, one can select on the basis of positive binding with the sema4D/CD100 polypeptide expressed by or isolated from cells of that species of animal.

The foregoing antibodies can be used in methods known in the art relating to the localization and activity of the sema4D/CD100 polypeptide (e.g., for Western blotting) sema4D/CD100 polypeptide in situ, measuring levels thereof in appropriate physiological samples, etc. using any of the detection techniques mentioned above or known in the art.

In a specific embodiment, antibodies that agonize or antagonize the activity of the sema4D/CD100 polypeptide can be generated. Such antibodies can be tested using the assays described infra for identifying ligands.

The invention also relates to single-chain Fv antibody fragments (ScFv) as described in U.S. Pat. No. 4,946,778 or by Martineau et al. (1998, J Mol Biol, 280(1):117-127).

The antibodies according to the invention also comprise antibody fragments obtained with the aid of phage libraries as described by Ridder et al., (1995, Biotechnology (NY), 13(3):255-260) or humanized antibodies as described by Reinmann et al. (1997, AIDS Res Hum Retroviruses, 13(11):933-943) and Leger et al., (1997, Hum Antibodies, 8(1):3-16).

The antibody preparations according to the invention are useful in immunological detection tests intended for the identification of the presence and/or of the quantity of antigens present in a sample.

An antibody according to the invention may comprise, in addition, a detectable marker which is isotopic or nonisotopic which are well known in the art, for example fluorescent, or may be coupled to a molecule such as biotin, according to techniques well known to persons skilled in the art.

In another aspect, the invention provides a method for identifying a test compound which modulates sema4D/CD100 activity, said method comprising: (a) contacting sema4D/CD100 with said test compound; (b) detecting a level of soluble sema4D/CD100 in the presence of the test compound and in the absence of the test compound; and (c) comparing said level of soluble sema4D/CD100 in the presence of said test compound with said level of soluble sema4D/CD100 in the absence of said test compound, wherein when a change in soluble level of sema4D/CD100 is detected between the level of soluble sema4D/CD100 in the presence of said test compound and the level of soluble sema4D/CD100 in the absence of said test compound, said test compound is determined to be a modulator of sema4D/CD100 activity.

The instant invention provides methods of treating a symptom of a platelet disorder and/or an endothelial cell disorder by modulating the level of soluble sema4D/CD100 polypeptide. As shown in the examples herein, there is an increase in sema4D/CD100 cleavage is in patients undergoing cardiopulmonary bypass and patients with heparin-induced thrombocytopenia or atherosclerosis. Therefore, in an embodiment, the method of decreasing the level of sema4D/CD100 would be advantageous. In another aspect, increasing the level of soluble sema4D/CD100 would be advantageous in order to increase platelet aggregation which would be useful in wound healing.

In one aspect, the invention provides a method of a method of identifying a compound that increases or reduces the level or activity of a sema4D/CD100 in a cell(s) comprising contacting a cell(s) with a test compound and comparing the level of soluble sema4D/CD100 or the activity of sema4D/CD100 in said cell with the level of soluble sema4D/CD100 or sema4D/CD100 activity in an otherwise identical cell(s) not contacted with the test compound, wherein a higher or lower level of soluble sema4D/CD100 or sema4D/CD100 activity in the cell(s) contacted with the test compound compared with the level of soluble sema4D/CD100 or sema4D/CD100 activity in the otherwise identical cell not contacted with the test compound is an indication that the test compound increases or reduces the sema4D/CD100 level or activity in a cell, thereby identifying a compound that increases or reduces the level or activity of the sema4D/CD100 in a cell. In an embodiment, the level of sema4D/CD100 is determined by measuring the level of Akt phosphorylation.

The invention further provides a method of identifying a therapeutic compound having activity to affect sema4D/CD100 by screening a test compound for its ability to modulate the expression or activity of sema4D/CD100. In an embodiment of the invention, a method includes analysis of the effect of a compound on sema4D/CD100 activity by comparing the result of: 1) contacting a cell comprising sema4D/CD100 with a test compound with the result obtained by 2) contacting a cell lacking sema4D/CD100 with the test compound. In an embodiment, a method includes providing a first cell comprising sema4D/CD100 and measuring Akt-dependent phosphorylation of the cell under defined culture conditions to obtain a phosphorylation value. Subsequently, the cell is contacted with the test compound and a second phosphorylation value is obtained. The difference between the first and second measured phosphorylation values provides an “inhibitory value,” which is a relative measure of the degree of inhibition of sema4D/CD100 when compared to the inhibitory value obtained by performing the method of the invention using a cell devoid of sema4D/CD100.

The difference in Akt-dependent phosphorylation between a sema4D/CD100-positive and a sema4D/CD100-negative cell, wherein the comparison is made both before and after treatment of the cells with a test compound, provides a relative measure of the effect of the test compound on sema4D/CD100. By way of a non-limiting example, a greater inhibitory value obtained by treating a sema4D/CD100-positive cell with a test compound than that obtained by treating a sema4D/CD100-negative cell with the test compound demonstrates a sema4D/CD100-inhibitory effect of the test compound. Methods of assaying for phosphorylation are known in the art, and are also described, in part, elsewhere herein.

In another embodiment, a method includes providing a first cell comprising sema4D/CD100 and measuring the amount of cleavage of sema4D/CD100 of the cell under defined culture conditions to obtain a cleavage value by measuring the amount of soluble sema4D/CD100. Subsequently, the cell is contacted with the test compound and a second cleavage value is obtained. The difference between the first and second measured cleavage values provides an “inhibitory value,” which is a relative measure of the degree of inhibition of sema4D/CD100 when compared to the inhibitory value obtained by performing the method of the invention using a cell devoid of sema4D/CD100.

The difference in sema4D/CD100 cleavage between a sema4D/CD100-positive and a sema4D/CD100-negative cell, wherein the comparison is made both before and after treatment of the cells with a test compound, provides a relative measure 6f the effect of the test compound on sema4D/CD100. By way of a non-limiting example, a greater inhibitory value obtained by treating a sema4D/CD100-positive cell with a test compound than that obtained by treating a sema4D/CD100-negative cell with the test compound demonstrates a sema4D/CD100-inhibitory effect of the test compound. Methods of assaying for protein cleavage products are known in the art, and are also described, in part, elsewhere herein.

Methods of the invention can be practiced in vitro, ex vivo or in vivo. When the method is practiced in vitro, the expression vector, protein or polypeptide can be added to the cells in culture or added to a pharmaceutically acceptable carrier as defined below. In addition, the expression vector or sema4D/CD100 DNA can be inserted into the target cell using well known techniques, such as transfection, electroporation or microinjection. By “target cell” is meant any cell that is the focus of examination, delivery, therapy, modulation or the like by, or as a result of, activation, inactivation, expression or changed expression of sema4D/CD100 or the nucleotide sequence encoding same, or any cell that effects such modulation, activation, inactivation or the like in the gene encoding it.

Compounds which are identified using the methods of the invention are candidate therapeutic compounds for treatment of disease states in patients caused by or associated with sema4D/CD100 or by a cell type related to the activation of sema4D/CD100, such as a platelet, endothelial cell, or a cell as yet unidentified which activates or is activated by the a condition in the subject, particularly in a human patient. By “patient” is meant any human or animal subject in need or treatment and/or to whom the compositions or methods of the present invention are applied. In a preferred embodiment of the invention, the patient is a mammal, more preferred that it is a veterinary animal, most preferred that it is a human.

The use of the methods in vitro provides a powerful bioassay for screening for drugs which are agonists or antagonists of sema4D/CD100 function in these cells. Thus, one can screen for drugs having similar or enhanced ability to prevent or inhibit sema4D/CD100 activity. It also is useful to assay for drugs having the ability to inhibit or activate platelet activation. The in vitro method further provides an assay to determine if the method of this invention is useful to treat a subject's pathological condition or disease that has been linked to enhanced sema4D/CD100 expression, to the developmental stages associated with up-regulation of sema4D/CD100, or to a platelet disorder.

Generally the term “activity,” as used herein, is intended to relate to sema4D/CD100 activity, for example the ability of sema4D/CD100 to enhance or inhibit Akt dependent phosphorylation, platelet activation and/or platelet aggregation, and an “effective amount” of a compound with regard to sema4D/CD100 activity means a compound that modulates (enhances or inhibits) sema4D/CD100 phosphorylation activity, platelet activation and/or platelet aggregation.

Acceptable “pharmaceutical carriers” are well known to those of skill in the art and can include, but are not limited to any of the standard pharmaceutical carriers, such as phosphate buffered saline, water and emulsions, such as oil/water emulsions and various types of wetting agents.

The assay method can also be practiced ex vivo. Generally, a sample of cells, such as platelets or endothelial cells, can be removed from a subject or animal using methods well known to those of skill in the art. An effective amount of antisense sema4D/CD100 nucleic acid or a sema4D/CD100 inhibitor or suspected sema4D/CD100 inhibitor is added to the cells and the cells are cultured under conditions that favor internalization of the nucleic acid by the cells. The transformed cells are then returned or reintroduced to the same subject or animal (autologous) or one of the same species (allogeneic) in an effective amount and in combination with appropriate pharmaceutical compositions and carriers.

As used herein, the term “administering” for in vivo and ex vivo purposes means providing the subject with an effective amount of the nucleic acid molecule or polypeptide effective to prevent, inhibit or enhance sema4D/CD100 phosphorylation activity, platelet activation and/or platelet aggregation in the target cell.

In each of the assays described, control experiments may include the use of mutant strains or cells types that do not encode sema4D/CD100. Such strains are generated by disruption of the sema4D/CD100 gene, generally in vitro, followed by recombination of the disrupted gene into the genome of host cell using technology which is available in the art of recombinant DNA technology as applied to the generation of such mutants in light of the present disclosure. The host may include transgenic hosts.

The invention also encompasses the use of pharmaceutical compositions of an appropriate antibody, protein or peptide, mimetope, peptidomimetic, and/or isolated nucleic acid to practice the methods of the invention, the compositions comprising an appropriate antibody, protein or peptide, mimetope, peptidomimetic, and/or isolated nucleic acid and a pharmaceutically-acceptable carrier.

As used herein, the term “pharmaceutically-acceptable carrier” means a chemical composition with which an appropriate antibody, protein or peptide, mimetope, peptidomimetic, and/or isolated nucleic acid may be combined and which, following the combination, can be used to administer the appropriate antibody, protein or peptide, mimetope, peptidomimetic, and/or isolated nucleic acid to a mammal.

The therapeutic methods of the invention thus encompass the use of pharmaceutical compositions of an appropriate small molecule, protein or peptide and/or isolated nucleic acid to practice the methods of the invention.

The pharmaceutical compositions useful for practicing the invention may be administered to deliver a dose of between 1 ng/kg/day and 100 mg/kg/day. In an embodiment, the invention envisions administration of a dose which results in a concentration of the compound of the present invention between 1 micromolar and 10 micromolar in a mammal.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers and AZT, protease inhibitors, reverse transcriptase inhibitors, interleukin-2,. interferons, cytokines, and the like.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In an embodiment of a formulation for parenteral administration, the active ingredient is provided in dry (i.e. powder or granular) form for reconstitution with a suitable vehicle (e.g. sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non toxic parenterally acceptable diluent or solvent as are well known in the art, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

The compound of the invention may also be assessed in non-transgenic animals to determine whether it acts through inhibition of sema4D/CD100 activity in vivo, or whether it acts via another mechanism. To test this effect of the test compound on activity, the procedures described above are followed using non-transgenic animals instead of transgenic animals.

This invention also provides vector and protein compositions useful for the preparation of medicaments which can be used for preventing or inhibiting sema4D/CD100 activity, maintaining cellular function and viability in a suitable cell, or for the treatment of a platelet disorder and/or endothelial disorder. In an embodiment, sema4D/CD100 activity is increased in order to promote wound healing by increasing platelet aggregation.

As used herein, “recombinant” is intended to mean that a particular DNA sequence is the product of various combination of cloning, restriction, and ligation steps resulting in a construct having a sequence distinguishable from homologous sequences found in natural systems. Recombinant sequences can be assembled from cloned fragments and short oligonucleotides linkers, or from a series of oligonucleotides.

The composition according to the invention is intended especially for the preventive or curative treatment of disorders, such as platelet disorders including but not limited to heart attack, stroke, thrombosis, thrombotic microangiopathy, disseminated intravascular coagulation, acquired platelet disorder and inherited platelet disorder; or by the activation of sema4D/CD100, or its homologue; or by expression or amplification of a presently unknown cell type, such as an epithelial cell, which is activated or transformed as a result of or related to sema4D/CD100 expression, or for which sema4D/CD100 expression is an indicator. The treatment of a platelet disorder (before or after the appearance of significant symptoms) is particularly preferred. The composition according to the invention is also intended to treat endothelial cell disorders including but not limited to thrombosis, aneurysm and arteriosclerosis.

The phrase “therapeutically effective amount” is used herein to mean an amount sufficient to reduce by at least about 15%, preferably by at least 50%, more preferably by at least 90%, and most preferably complete remission of a hyperproliferative disease or cancer of the host. Alternatively, a “therapeutically effective amount” is sufficient to cause an improvement in a clinically significant condition in the host. In the context of the present invention, a therapeutically effective amount of the expression product of sema4D/CD100 or a homologue thereof, particularly the human homologue, is that amount which is effective to treat a platelet disorder, in a patient or host, thereby effecting a reduction in size or virulence or the elimination of such disease. Preferably, administration or expression of an “effective” amount of the expression product of sema4D/CD100 or a homologue thereof, particularly the human homologue resolves the underlying disease.

In another aspect, the invention provides a method for identifying a test compound which modulates sema4D/CD100 activity, said method comprising: (a) contacting sema4D/CD100 with said test compound; (b) detecting a level of soluble sema4D/CD100 in the presence of the test compound and in the absence of the test compound; and (c) comparing said level of soluble sema4D/CD100 in the presence of said test compound with said level of soluble sema4D/CD100 in the absence of said test compound, wherein when a change in soluble level of sema4D/CD100 is detected between the level of soluble sema4D/CD100 in the presence of said test compound and the level of soluble sema4D/CD100 in the absence of said test compound, said test compound is determined to be a modulator of sema4D/CD100 activity.

The present invention further includes a method of identifying a compound that affects expression of sema4D/CD100 in a cell. The method comprises contacting a cell with a test compound and comparing the level of expression of sema4D/CD100 in the cell so contacted with the level of expression of sema4D/CD100 in an otherwise identical cell not contacted with the compound. If the level of expression of sema4D/CD100 is higher or lower in the cell contacted with the test compound compared to the level of expression of sema4D/CD100 in the otherwise identical cell not contacted with the test compound, this is an indication that the test compound affects expression of sema4D/CD100 in a cell.

One skilled in the art would appreciate, based upon the disclosure provided herein, that assessing the level of sema4D/CD100 can be performed using probes (e.g., antibodies and/or nucleic acid probes that specifically bind with sema4D/CD100 gene or protein), such that the method can identify a compound that selectively affects expression of sema4D/CD100. Such compounds are useful for inhibiting expression of sema4D/CD100. One skilled in the art would understand that such compounds can be useful for inhibiting a disease, disorder, or condition mediated by and/or associated with increased expression of sema4D/CD100, e.g., increased levels of sema4D/CD100 is associated with platelet disorders and/or endothelial cell disorders. Also, the levels of sema4D/CD100 activity by cells can be measured by evaluation of Akt phosphorylation. Thus, the skilled artisan would appreciate, based on the disclosure provided herein, that it may useful to decrease expression of sema4D/CD100 and/or the soluble level of sema4D/CD100.

The skilled artisan will further appreciate that the present invention is not limited to a method of identifying a useful compound in a cell or an animal. That is, the present invention provides methods of identifying a useful compound in a cell-free system. A cell-free system, as used herein, refers to an in vitro assay wherein the components necessary for a reaction to take place are present, but are not associated with a cell. Such components can include cellular enzymes, transcription factors, proteins, nucleic acids, and the like, provided that they are substantially free from a cell. Thereby, the present invention provides a method of identifying a useful compound for treating platelet disorders and/or endothelial cell disorders and preventing the progression of platelet disorders and/or endothelial cell disorders or an associated disease or condition.

One skilled in the art would appreciate, based on the disclosure provided herein, that the level of expression of sema4D/CD100 in the cell may be measured by determining the level of expression of mRNA encoding sema4D/CD100 or sema4D/CD100 protein itself. Alternatively, the level of expression of sema4D/CD100 can be determined by using immunological methods to assess sema4D/CD100 production using anti-sema4D/CD100 antibodies. Further, nucleic acid-based detection methods, such as Northern blot and PCR assays and the like, can be used as well. In addition, the level of sema4D/CD100 activity in a cell can also be assessed by determining the level of various parameters which can be affected by sema4D/CD100 activity, such as, for example, Akt phosphorylation. Thus, one skilled in the art would appreciate, based upon the disclosure and reduction to practice provided herein, that there are a multitude of methods that are well-known in the art which can be used to assess the level of sema4D/CD100 activity in a cell including those disclosed herein and others which may be developed in the future.

In addition, a protein that specifically binds with sema4D/CD 100 can be identified using, for example, a yeast two hybrid assay. Yeast two hybrid assay methods are well-known in the art and can be performed using well documented techniques, for example those described in Bartel and Fields, (The Yeast Two-Hybrid System, Oxford University Press, Cary, N.C.). Therefore, once armed with the teachings provided herein, e.g., the full amino and nucleic acid sequences of the sema4D/CD100 protein, one skilled in the art can easily identify a protein that specifically binds with sema4D/CD100.

One skilled in the art would understand, based upon the disclosure provided herein, that the invention encompasses any molecule identified using the methods discussed elsewhere herein. That is, molecules that associate with sema4D/CD100 or an sema4D/CD100 target protein, can be used to develop therapeutics and diagnostics for diseases, disorders or conditions mediated by sema4D/CD100. That is, one skilled in the art would appreciate, as more fully set forth elsewhere herein in discussing antibodies that specifically bind with sema4D/CD100, that a sema4D/CD100-associated protein can be used to develop therapeutics that inhibit sema4D/CD100 activity in a cell by inhibiting sema4D/CD100 expression or inhibiting cleavage of sema4D/CD100 and, therefore, sema4D/CD100 binding interactions and/or sema4D/CD100 activity.

Sema4D/CD100-associated proteins identified by the above-disclosed methods can be used directly to inhibit sema4D/CD100 interactions by contacting a cell with the sema4D/CD100-associated protein, or a portion thereof, or they can be used to develop antibodies and/or peptidomimetics that can inhibit the sema4D/CD100-associated protein interaction with sema4D/CD100 thereby inhibiting sema4D/CD100 function, activity, and cleavage. Thus, sema4D/CD100-associated proteins are useful and are encompassed by the invention. In an embodiment, a test compound of the invention is inhibits the binding of sema4D/CD100 to its receptor. In a preferred embodiment, the receptor is CD72 or plexin B 1.

The present invention also relates to cloning vectors containing a gene encoding analogs and derivatives of the sema4D/CD100 polypeptide and/or an sema4D/CD100 binding sequence of the invention. The production and use of derivatives and analogs related to the sema4D/CD100 protein are within the scope of the present invention. In a specific embodiment, the derivatives or analogs are functionally active, e.g., capable of exhibiting one or more functional activities associated with a full-length, wild-type sema4D/CD100 polypeptide of the invention.

Due to the degeneracy of nucleotide coding sequences, other DNA sequences which encode substantially the same amino acid sequences as that of sema4D/CD100 gene may be used in the practice of the present invention. These include but are not limited to allelic genes, homologous genes from other species, and nucleotide sequences comprising all or portions of sema4D/CD100 gene which are altered by the substitution of different codons that encode the same amino acid residue within the sequence, thus producing a silent change. Likewise, the sema4D/CD100 derivatives of the invention include, but are not limited to, those containing, as a primary amino acid sequence, all or part of the amino acid sequence of the sema4D/CD100 protein including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs.

A large number of vector-host systems known in the art may be used. Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Possible vectors include, but are not limited to, plasmids or modified viruses, but the vector system must be compatible with the host cell used. Examples of vectors include, but are not limited to, Escherichia coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, infection, electroporation, etc., so that many copies of the gene sequence are generated. The cloned gene may be contained on a shuttle vector plasmid, which provides for expansion in a cloning cell, e.g., Escherichia coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences from the yeast 2 m plasmid.

In an alternative method, the desired gene may be identified and isolated after insertion into a suitable cloning vector in a “shot gun” approach. Enrichment for the desired gene, for example, by size fractionation, can be done before insertion into the cloning vector.

The nucleotide sequence coding for the sema4D/CD100 polypeptide or antigenic fragments, derivatives or analogs thereof, or functionally active derivatives, including chimeric proteins thereof, may be inserted into an appropriate expression vector, e.g., a vector which contains the necessary elements for the transcription and translation of the inserted protein-coding sequence. Such elements are termed herein a “promoter.” Thus, nucleic acid encoding the sema4D/CD100 polypeptide of the invention are operationally associated with a promoter in an expression vector of the invention. Both cDNA and genomic sequences can be cloned and expressed under control of such regulatory sequences. An expression vector may also include a replication origin.

The necessary transcriptional and translational signals can be provided on a recombinant expression vector, or they may be supplied by a native gene encoding sema4D/CD100 and/or its flanking regions.

A recombinant sema4D/CD100 protein of the invention, or functional fragments, derivatives, chimeric constructs, or analogs thereof, may be expressed chromosomally, after integration of the coding sequence by recombination. In this regard, any of a number of amplification systems may be used to achieve high levels of stable gene expression (See Sambrook et al., 2001, supra).

The cell into which the recombinant vector comprising the nucleic acid encoding the sema4D/CD100 polypeptide or an sema4D/CD100 binding sequence is cultured in an appropriate cell culture medium under conditions that provide for expression of the sema4D/CD100 polypeptide or an sema4D/CD100 binding sequence by the cell.

Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

Expression of the sema4D/CD100 polypeptide or an sema4D/CD100 binding sequence maybe controlled by any promoter/enhancer element known in the art, but these regulatory elements must be functional in the host selected for expression. Promoters which may be used to control sema4D/CD100 gene expression include, but are not limited to, the SV40 early promoter region (Benoist and Chambon, 1981 Nature 290:304-310), the promoter contained in the 3′ long terminal repeat of Rous sarcoma virus (Yamamoto, et al., 1980 Cell 22:787-797), the herpes thymidine kinase promoter (Wagner et al., 1981 Proc. Natl. Acad. Sci. U.S.A. 78:1441-1445), the regulatory sequences of the metallothionein gene (Brinster et al., 1982 Nature 296:39-42); prokaryotic expression vectors such as the β-lactamase promoter (Villa-Kamaroff, et al., 1978 Proc. Natl. Acad. Sci. U.S.A. 75:3727-3731), or the Tac promoter (DeBoer, et al., 1983 Proc. Natl. Acad. Sci. U.S.A. 80:21-25); see also “Useful proteins from recombinant bacteria” in Scientific American, 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., 1984 Cell 38:639-646; Ornitz et al., 1986 Cold Spring Harbor Symp. Quant. Biol. 50:399-409; MacDonald, 1987); insulin gene control region which is active in pancreatic beta cells (Hanahan, 1985 Nature: 315:115-122), immunoglobulin gene control region which is active in lymphoid cells (Grosschedl et al., 1984 Cell 38:647-658; Adames et al., 1985 Nature 318:533-538; Alexander et al., 1987 Mol. Cell. Biol. 7:1436-1444), mouse mammary tumor virus control region which is active in testicular, breast, lymphoid and mast cells (Leder et al., 1986 Cell 45:485-495), albumin gene control region which is active in liver (Pinkert et al., 1987 Genes and Devel. 1:268-276), alpha-fetoprotein gene control region which is active in liver (Krumlauf et al., 1985 Mol. Cell. Biol. 5:1639-1648; Hammer et al., 1987 Science 235:53-58), alpha 1-antitrypsin gene control region which is active in the liver (Kelsey et al., 1987 Genes and Devel. 1:161-171) beta-globin gene control region which is active in myeloid cells (Mogram et al., 1985 Nature 315:338-340; Kollias et al., 1986 Cell 46:89-94), myelin basic protein gene control region which is active in oligodendrocyte cells in the brain (Readhead et al., 1987 Cell 48:703-712), myosin light chain-2 gene control region which is active in skeletal muscle (Sani, 1985 Nature 314:283-286), and gonadotropic releasing hormone gene control region which is active in the hypothalamus (Mason et al., 1986 Science 234:1372-1378).

Expression vectors containing a nucleic acid encoding the sema4D/CD100 polypeptide or sema4D/CD100 binding sequence of the invention can be identified by five general approaches: (a) polymerase chain reaction (PCR) amplification of the desired plasmid DNA or specific mRNA, (b) nucleic acid hybridization, (c) presence or absence of selection marker gene functions, (d) analyses with appropriate restriction endonucleases, and (e) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In another example, if the nucleic acid encoding the sema4D/CD100 polypeptide or an sema4D/CD100 binding sequence is inserted within the “selection marker” gene sequence of the vector, recombinants containing the sema4D/CD100 nucleic acid can be identified by the absence of the sema4D/CD100 gene functions. In the fourth approach, recombinant expression vectors are identified by digestion with appropriate restriction enzymes. In the fifth approach, recombinant expression vectors can be identified by assaying for the activity, biochemical, or immunological characteristics of the gene product expressed by the recombinant, provided that the expressed protein assumes a functionally active conformation.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR; See, Kaufman, Current Protocols in Molecular Biology, 16.12 (1991). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). Yeast expression systems can also be used according to the invention to express the sema4D/CD100 polypeptide. For example, the non-fusion pYES2 vector (XbaI, SphI, ShoI, NotI, GstXI, EcoRI, BstXI, BamH1, SacI, KpnI, and HindIII cloning sit; Invitrogen) or the fusion pYESHisA, B, C (XbaI, SphI, ShoI, NotI, BstXI, EcoRI, BamHII, SacI, KpnI, and HindIII cloning site, N-terminal peptide purified with ProBond resin and cleaved with enterokinase; Invitrogen), to mention just two, can be employed according to the invention.

In addition, a host cell strain may be chosen which modulates the expression of the inserted sequences, or modifies and processes the gene product in the specific fashion desired different host cells have characteristic and specific mechanisms for the translational and post-translational processing and modification (e.g., glycosylation, cleavage for example of the signal sequence) of proteins. Appropriate cell lines or host systems can be chosen to ensure the desired modification and processing of the foreign protein expressed. For example, expression in a bacterial system can be used to produce an nonglycosylated core protein product. However, the transmembrane sema4D/CD100 protein expressed in bacteria may not be properly folded. Expression in yeast can produce a glycosylated product. Expression in eukaryotic cells can increase the likelihood of “native” glycosylation and folding of a heterologous protein. Moreover, expression in mammalian cells can provide a tool for reconstituting, or constituting, sema4D/CD100 activity. Furthermore, different vector/host expression systems may affect processing reactions, such as proteolytic cleavages, to a different extent.

Vectors are introduced into the desired host cells by methods known in the art, e.g., transfection, electroporation, microinjection, transduction, cell fusion, DEAE Dextran, calcium phosphate precipitation, lipofection (lysosome fusion), use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

A cell has been “transfected” by exogenous or heterologous DNA when such DNA has been introduced inside the cell. A cell has been “transformed” by exogenous or heterologous DNA when the transfected DNA effects a phenotypic change. The transforming DNA may be integrated (covalently linked) into chromosomal DNA making up the genome of the cell.

A recombinant protein such as sema4D/CD100 which is expressed as an integral membrane protein can be isolated and purified by standard methods. Generally, the integral membrane protein can be obtained by lysing the membrane with detergents, such as but not limited to, sodium dodecyl sulfate (SDS), Triton X-100 polyoxyethylene ester, Ipagel/nonidet P-40 (NP-40) (octylphenoxy)-polyethoxyethanol, Digoxin, sodium deoxycholate, and the like, including mixtures thereof. Solubilization can be enhanced by sonication of the suspension. Soluble forms of the protein can be obtained by collecting culture fluid, or solubilizing inclusion bodies, e.g., by treatment with detergent, and if desired sonication or other mechanical processes, as described above. The solubilized or soluble protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

Alternatively, a nucleic acid or vector according to the invention can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Felgner, et. al. (1987. PNAS 84/7413); Mackey, et al. (1988, Proc. Natl. Acad. Sci. USA, 85 :8027-8031); Ulmer et al. (1993, Science, 259 :1745-1748). The use of cationic lipids may promote encapsulation of negatively charged nucleic acids, and also promote fusion with negatively charged cell membranes (Felgner et al., 1989, Science, 337:387-388). Particularly useful lipid compounds and compositions for transfer of nucleic acids are described in International Patent Publications WO95/18863 and WO96/17823, and in U.S. Pat. No. 5,459,127. The use of lipofection to introduce exogenous genes into the specific organs in vivo has certain practical advantages. Molecular targeting of liposomes to specific cells represents one area of benefit. It is clear that directing transfection to particular cell types would be particularly useful in a tissue with cellular heterogeneity, such as pancreas, liver, kidney, and the brain. Lipids may be chemically coupled to other molecules for the purpose of targeting [see Mackey, et. al., supra]. Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., International Patent Publication WO95/21931), peptides derived from DNA binding proteins (e.g., International Patent Publication WO96/25508), or a cationic polymer (e.g., International Patent Publication WO95/21931).

It is also possible to introduce the vector in vivo as a naked DNA plasmid (see U.S. Pat. Nos. 5,693,622, 5,589,466 and 5,580,859). Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, Wu et al., 1992, supra; Wu and Wu, 1988, supra; Hartmut et al., Canadian Patent Application No. 2,012,311, filed Mar. 15, 1990; Williams et al., 1991, Proc. Natl. Acad. Sci. USA 88:2726-2730). Receptor-mediated DNA delivery approaches can also be used (Curiel et al., 1992, Hum. Gene Ther. 3:147-154; Wu and Wu, 1987, J. Biol. Chem. 262:4429-4432).

The present invention also provides kits useful in practicing the methods of the invention.

The present invention encompasses various kits which comprise a compound, including a nucleic acid encoding sema4D/CD100, an sema4D/CD100 polypeptide, an antibody that specifically binds sema4D/CD100, a nucleic acid complementary to a nucleic acid encoding sema4D/CD100 but in an antisense orientation, an applicator, and instruction manual which describe use of the compound to perform the methods of the invention. Although model kits are described below, the contents of other useful kits will be apparent to the skilled artisan in light of the present disclosure. Each of these kits is contemplated within the present invention.

In one aspect, the invention provides a kit for treating a platelet disorder and/or endothelial cell disorder and preventing the development of a platelet disorder and/or endothelial cell disorder and/or associated diseases or conditions. The kit is used in the same manner as the methods disclosed herein for the present invention. Briefly, the kit may be used to contact a cell with a nucleic acid encoding an sema4D/CD100 molecule of the invention. Additionally, the kit comprises an applicator and an instruction manual for the use of the kit. These instructions simply embody the examples provided herein.

The kit further includes a pharmaceutically-acceptable carrier. The composition is provided in an appropriate amount as set forth elsewhere herein. Further, the route of administration and the frequency of administration are as previously set forth elsewhere herein.

In another aspect, the invention provides a kit for treating platelet disorders and/or endothelial cell disorders and/or associated diseases or conditions. The kit is used in the same manner as the methods disclosed herein for the present invention. Briefly, the kit may be used to contact a cell with an inhibitor or an activator of sema4D/CD100. Additionally, the kit comprises an applicator and an instruction manual for the use of the kit. These instructions simply embody the examples provided herein. The kit further includes a pharmaceutically-acceptable carrier. The composition is provided in an appropriate amount as set forth elsewhere herein. Further, the route of administration and the frequency of administration are as previously set forth elsewhere herein.

The invention further encompasses a kit for the treatment of a platelet disorder and/or an endothelial cell disorder and the prevention of a platelet disorder and/or endothelial cell disorder and/or associated diseases or conditions. The skilled artisan will appreciate that the kit can be used according to the methods set forth herein. The kit comprises an antibody, small molecule, or peptide that binds sema4D/CD100, or some fragment thereof, an applicator, and an instruction manual substantially similar to the examples provided herein. The kit further includes a pharmaceutically acceptable carrier, of which the composition, route of administration, and frequency of administration are as previously disclosed elsewhere herein.

Further, the invention comprises a kit for treating a platelet disorder and/or endothelial cell disorder and/or associated diseases or conditions comprising an antisense nucleic acid complementary to a nucleic acid encoding a mammalian sema4D/CD100 molecule, or some fragment thereof. Such kits can be used according to the methods of the invention to mediate the decreased expression of sema4D/CD100. Additionally, the kit comprises an applicator and an instruction manual for the use of the kit. These instructions simply embody the examples provided herein. The kit further includes a pharmaceutically-acceptable carrier. The antisense nucleic acid and pharmaceutically-acceptable carrier are provided in an appropriate amount as set forth elsewhere herein. Further, the route of administration and the frequency of administration are as previously set forth elsewhere herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLE 1

### Sema4D/CD100 is Expressed in Resting Human Platelets

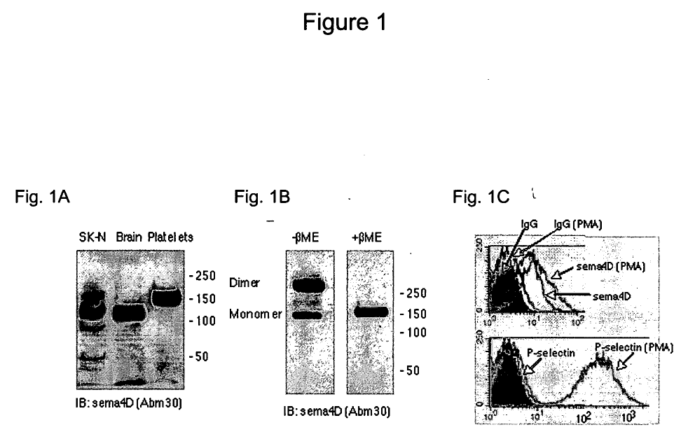

The initial screen for semaphorin family members showed that human and mouse platelets express sema4D/CD100, showing up on immunoblots as a prominent band of 150 kDa under reducing conditions and 300 kDa under non-reducing conditions (FIG. 1). The size difference presumably reflects the formation of a disulfide-linked homodimer [18], but it is worth noting that the apparent size of sema4D/CD100 in platelets is somewhat larger than the dominant band detected in human brain, human neuroblastoma SK-N-SH cells (FIG. 1A) or Jurkat cells (not shown). The antibody used in FIG. 1A, Abm30, is directed against the intracellular C-terminus of sema4D/CD100, but a band of identical size was seen in precipitates obtained with antibodies directed against the extracellular domain (not shown). Incubation with the endoglycosidase, PGNase F, caused a decrease in the apparent size of platelet sema4D/CD100, but did not fully eliminate the observed size differences. However, reverse transcription and amplification (RT-PCR) of RNA from platelets, brain, HEL cells and Jurkat cells using primers encompassing the entire coding sequence of sema4D/CD100 produced sets of fragments that were of identical length in each case and the sequence of sema4D/CD100 RNA amplified from platelets matched that which has been reported previously. This suggests that the size differences are due to post-translational modifications, including glycosylation, rather than differences in sequence.

Surface expression of sema4D/CD100 was detected by FACS using an antibody that recognizes the extracellular domain. Expression increased when the platelets were activated with PMA for 15 minutes (FIG. 1C). In contrast, the α-granule membrane protein, P-selectin, was detected only on activated platelets. Taken together, these results show that resting human platelets express sema4D/CD100 as a glycosylated disulfide-linked homodimer and that platelet activation can cause an increase in sema4D/CD100 surface expression.

### EXAMPLE 2

### Regulated Recruitment and Shedding of sema4D/CD100 During Platelet Activiation

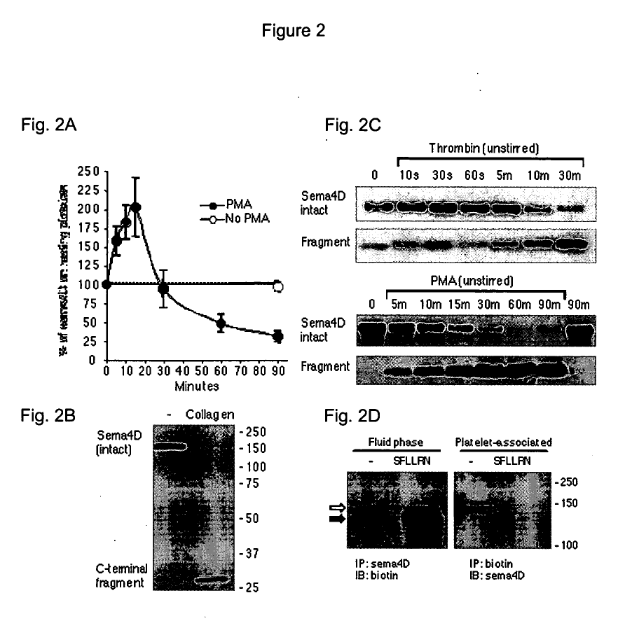

FIG. 2A follows change in sema4D/CD100 surface expression over time. The results show that after the initial increase in expression noted in FIG. 1C, surface expression gradually declines to essentially undetectable levels. This loss of sema4D/CD100 surface expression may be due to cleavage of the protein or internalization (or both). To test these possibilities, washed platelets were stimulated with collagen and allowed to aggregate. Immunoblots showed loss of the 150 kDa full-length protein and the appearance of a 25-30 kDa C-terminal fragment that was not present in resting platelets (FIG. 2B). Time course studies performed under non-aggregating conditions showed that two other agonists, thrombin and PMA, also caused a time-dependent loss of the intact protein with the concurrent appearance of a 25-30 kDa C-terminal fragment that was detectable shortly agonist addition. This fragment remained with the platelets and presumably includes the transmembrane domain as well as the intracellular domain recognized by the antibody. The intact 150 kDa protein was typically undetectable after 30-60 minutes (FIG. 2C).

To determine whether the sema4D/CD100 extracellular domain is shed from the platelet surface, resting platelets were incubated with a non-permeable biotinylation reagent (biotin-7-NHS) and then stimulated with the PAR-1 (thrombin receptor) agonist peptide, SFLLRN. Biotinylated sema4D/CD100 was immunoprecipitated from the fluid phase using an antibody directed against the sema4D/CD100 N-terminus and then immunoblotted with an anti-biotin antibody. The results in FIG. 2D show that an ˜130 kDa fragment of the sema4D/CD100 extracellular domain (filled arrow) is present in the supernate of activated platelets, but not resting platelets. In parallel, anti-biotin immunoprecipitates from platelet lysates show a decrease in the amount of intact, platelet-associated sema4D/CD100 (open arrow) when the platelets were activated. Taken together, these data show that sema4D/CD100 undergoes regulated recruitment to the platelet surface in response to agonists, after which the extracellular domain is gradually cleaved and shed, accounting for the loss of sema4D/CD100 surface expression. There is at least one large exodomain fragment produced by the cleavage event, leaving behind a 25-30 kDa fragment that includes the transmembrane and cytoplasmic domains.

### EXAMPLE 3

### Platelet Aggregation Enhances the Rate of sema4D/CD100 Cleavage

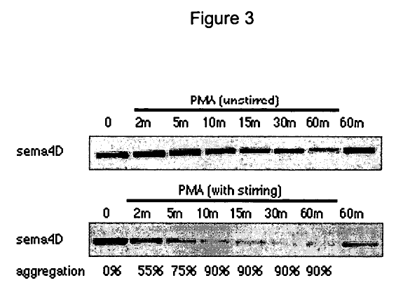

To address the issue of whether the rate of sema4D/CD100 cleavage is different when aggregation is allowed to occur than when activation occurs in the absence of aggregation, platelets were activated with or without the stirring needed for platelet aggregates to form. PMA caused a time-dependent loss of intact sema4D/CD100 and the appearance of the C-terminal sema4D/CD100 fragment. The rate of cleavage was accelerated when aggregation was allowed to occur (FIG. 3). Even at its fastest, however, cleavage lagged behind aggregation. The observed acceleration of cleavage required more than just stirring the platelets: when the platelets were stirred, but fibrinogen binding and, therefore, aggregation was inhibited by adding the peptide, RGDS, the rate of cleavage became more like that seen when the platelets were activated without stirring (not shown). These results show that sema4D/CD100 cleavage is accelerated during platelet aggregation.

### EXAMPLE 4

### Cleavage of sema4D/CD100 is Prevented by Metalloprotease Inhibitors

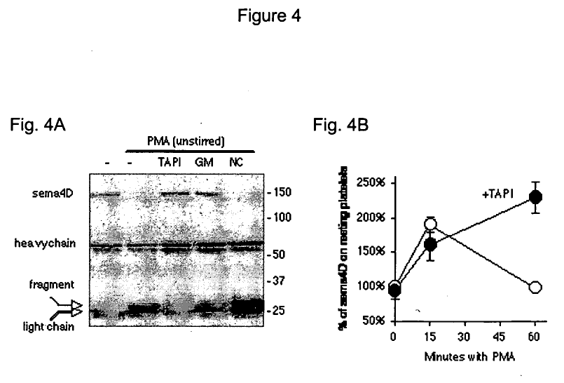

The observation that sema4D/CD100 cleavage occurs in washed platelets implies that the protease involved is intrinsic to the platelets and not a plasma protein. Platelets are known to contain a number of metalloproteases, including members of the MMP and ADAM families (Sheu, J. R., et al., Br J Pharmacol, 2004. 143(1): p. 193-201. Sawicki, G., et al., Nature, 1997. 386: p. 616-619. Sawicki, G., et al., Thromb. Haemost., 1998. 80: p. 836-839. Colciaghi, F., et al., Mol Med, 2002. 8(2): p. 67-74.). Consistent with a role for these proteases, it was found that cleavage was inhibited by EDTA (not shown) and the metalloprotease inhibitors, TAPI-2 (Rowe, P. D., et al., J Exp Med, 1995. 181(3): p. 1205-10) and GM6001 (Galardy, R. E., et al., Matrix Suppl, 1992. 1: p. 259-62) (FIG. 4A). TAPI-2 also abolished sema4D/CD100 cleavage in platelets activated by thrombin or SFLLRN (not shown), and prevented the loss of surface expression otherwise seen when platelets were stimulated with PMA (FIG. 4B). The initial increase in sema4D/CD100 surface expression still occurred; the subsequent loss did not. Thus, these results show that cleavage of sema4D/CD100 is prevented by metalloprotease inhibitors.

### EXAMPLE 5

### Identification of ADAM17/TACE as a Metalloprotease Required for Sema4D/CD100 Cleavage

Sema4D/CD100 is not the only protein that undergoes regulated shedding during platelet activation. Other examples include glycoprotein (GP) Ibα, GP V, GP VI and CD40L. Although for the most part the proteases involved have not been identified, recent reports show that ADAM17 is required for cleavage of GP Ibα (Bergmeier, W., et al., Circ Res, 2004. 95(7): p. 677-83) and GPV (Rabie, T., et al., J Biol Chem, 2005). ADAM17 is a zinc-dependent metalloprotease in with a disintegrin and a metalloprotease domain (Black, R. A. and J. M. White, ADAMs: 10: p. 654-659). ADAM17 is synthesized as an inactive precursor. Cleavage of the prodomain renders it active. ADAM17 (or TACE) was originally identified as the protease that cleaves TNFα (ack, R. A., et al., Nature, 1997.385: p. 729-733), but it has other substrates as well (Arribas, J. and A. Borroto, Chem Rev, 2002. 102(12): p. 4627-38).

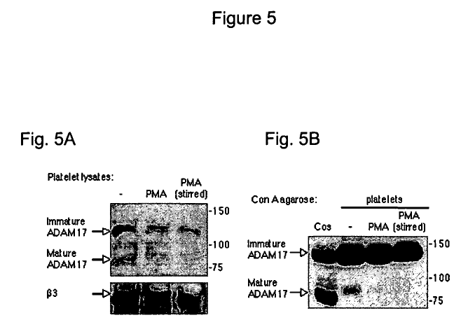

To test the involvement of ADAM17 in sema4D/CD100 cleavage, the expression of ADAM17 was first evaluated in platelets. Immunoblots of platelet lysates with a monoclonal antibody against the catalytic domain of ADAM17 (Tc3-7.49) showed the zymogen (or “immature”) form of ADAM17 in both resting and activated platelets (FIG. 5A). This is consistent with an immunoblot using a different ADAM17 antibody reported by Rabie, et al. (Rabie, T., et al., J Biol Chem, 2005). In addition to the zymogen, a band corresponding in size to the mature (active) form of ADAM17 was readily detected in COS-7 cells, but was much less abundant in platelets relative to the amount of zymogen, even when platelet glycoproteins were concentrated by affinity chromatography with concanavalin A (FIG. 5B). After a 30 minute incubation with PMA, the mature form of ADAM17 was still detectable, but further diminished.

### EXAMPLE 6

### Role of sema4D/CD100 in Platelet Aggregation

Blood was isolated from the inferior vena cava of anesthetized mice (100 mg/kg pentobarbital) using a heparinized syringe (15 units/ml blood), diluted 1:1 with HEPES-Tyrode's buffer, and spun at 100×g for 7 minutes to remove red cells. The final platelet count was adjusted to 4×108/ml with platelet poor plasma (PPP) from the same mouse. Aggregation was initiated with 2.5 μl of agonist applied to a 250 μl aliquot of PRP (platelet rich plasma) and was measured in a ChronoLog lumi-aggregometer (Havertown, Pa.). The agonists that were used include ADP, collagen, a TxA2 analog (U46619) and a PAR4 thrombin receptor agonist peptide (AYPGQV). Whenever appropriate, limited or full dose/response curves were measured.

Platelet aggregation study also included the use of washed platelets were stimulated with 100 nM PMA in an aggregometer cuvette at 37° C. with or without stirring. Lysates were precipitated and blotted with antibody Abm30.

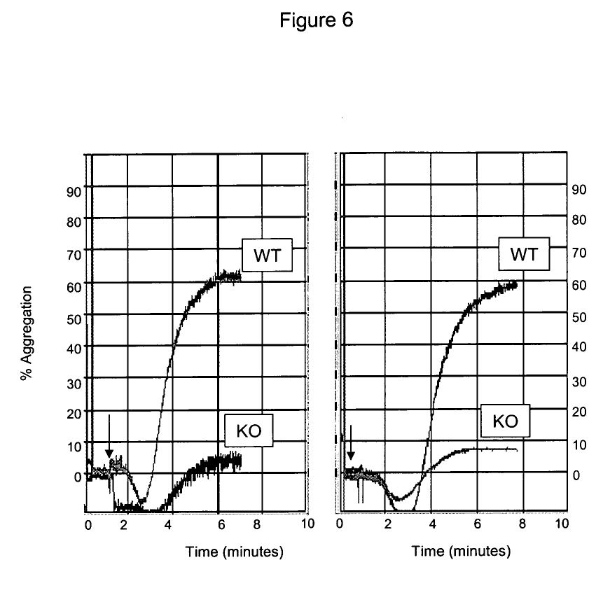

Collagen-stimulated platelet aggregation was examined using platelets from the sema4D/CD100 knockout (−/−) mice. The controls are age and gender-matched mice, either from the same or a closely-related het X het cross. The results show a clear decrease in the extent of aggregation in the sema4D/CD100 (−/−) platelets (FIG. 6). In contrast, platelets that lack CD72 show increased responsiveness in the absence of CD72 (data not shown). Both of these observations show that an interaction between sema4D and CD72 releases the tonic inhibitory effect produced by the association of CD72 with SHP-1.

### EXAMPLE 7

### PI 3-kinase and Akt-dependent Signaling is Impaired in sema4D Knockout Mice

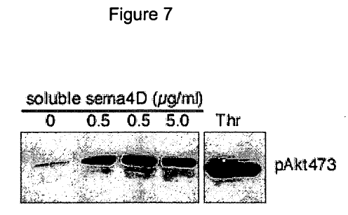

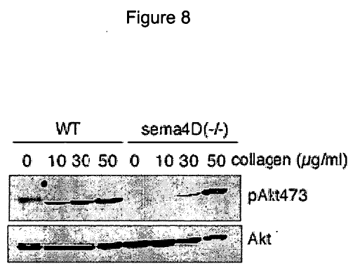

As shown in FIGS. 7 and 8, PI 3-kinase and Akt-dependent signaling was also impaired in platelets that lack sema4D/CD100. Activation of the PI 3-kinase pathway was measured using phosphorylation of Akt as a surrogate. Akt phosphorylation normally occurs in platelets activated by collagen and other agonists. Akt phosphorylation was also recently shown to occur in endothelial cells stimulated with soluble sema4D/CD100 (Basile, et al. Mol Cell Biol. 16: 6889, 2005). In endothelial cells responses to sema4D/CD100 appear to be mediated by plexin-B1. In FIG. 7, human platelets were stimulated with a His-tagged protein corresponding to the human sema4D/CD100 exodomain. Platelet lysates were prepared and immunoblotted with an antibody specific for pAkt473. Thrombin was used as a positive control. The results show that pAkt is absent in resting platelets, but readily detectable in the platelets stimulated with thrombin or soluble sema4D/CD100. Thus, these results show that the PI3K/Akt pathway plays a role in downstream events that involve sema4D/CD100 and its receptors.

If sema4D/CD100 promotes signaling pathways in platelets that involve Akt phosphorylation, then loss of sema4D/CD100 should impair Akt phosphorylation. In FIG. 8, collagen-stimulated Akt phosphorylation was compared in platelets from sema4D/CD100 (−/−) mice and matched controls. The results show that Akt phosphorylation was inhibited in the platelets from the sema4D/CD100 (−/−) mice.

### EXAMPLE 8

### Characterization of the Role of sema4D/CD100 in Thrombosis

Dense Granule Secretion

PRP is pooled from two mice of each genotype, incubated for 30 minutes at 37□C with (3H)5-HT (5-hydroxytryptamine; serotonin) (1 μCi/ml), and washed once with HEN containing 1 μM imipramine, then resuspended in HEPES-Tyrode's containing 1 μM imipramine and 1 mM CaCl2. Platelets (4×108/ml) are stimulated (in 200 μl aliquots) with the indicated concentrations of AYPGQV for 10 minutes at 37° C. Reactions are stopped with an equal volume of 0.1 M EDTA/2% formaldehyde, and centrifuged for 10 minutes at 900×g. The percentage of 5-HT release is defined as the agonist-related increase in extracellular 3H divided by the total intracellular ³H at the start of the experiment.

α-Granule Secretion and Fibrinogen Binding

To measure platelet α-granule secretion, washed mouse platelets (1×108/ml) are incubated simultaneously with 1 μg/ml FITC-labeled anti-mouse-P-selectin antibody (Pharmingen, San Diego, Calif., USA) and indicated concentrations of agonist peptide AYPGQV at 37° C. for 20 minutes. The platelets are then fixed in 1% formalin-containing Tyrode's buffer for 10 minutes at 37° C., diluted 5× with Tyrode's buffer and analyzed by flow cytometry. To measure fibrinogen binding, platelets are treated as above, but incubated with FITC-labeled mouse fibrinogen (100 μg/ml) instead of P-selectin antibody.

Carotid Artery Injury Model of Thrombosis

After anesthesia with sodium pentobarbital 100 mg/kg, the exposed carotid artery of an adult mouse (6-10 weeks of age, 18-30 g) is placed in contact with a strip of filter paper soaked with 10% FeCl3 for 2.5 minutes before being rinsing with phosphate-buffered saline (PBS). Blood flow in the artery is recorded for 30-60 minutes using a Doppler flow probe with digital data output captured in a PC. As an alternative to the full-thickness injury cause by FeCl3, there is still the ability to cause a laser-induced injury after infusing rose Bengal dye.

Tail Bleeding Times

Tails of anesthetized mice (Pentobarbital 100 mg/kg) (6-10 weeks of age, 18-30 g) are transected 1 mm from the tip, the remaining tail is immersed in 37□C saline and the time until bleeding stopped for more than one minute is recorded. The amount of blood loss is determined by measuring the optical density of the saline at the end of the period.

Disseminated Thrombosis

The mice receive a 100 μl tail vein injection of either 1) collagen (150 μg/ml, ChronoLog, Havertown, Pa.) plus epinephrine (300 μM) or 2) collagen (150 μg/ml) plus ADP (170 mM) by tail vein injection. The endpoint is mortality over 10 minutes. In some experiments, the lungs are removed immediately after death, fixed with 4% paraformaldehyde, embedded and then sectioned. The sections are stained with H&E and examined for the extent of thrombosis.

Since it has already been established that sema4D/CD100 is present in both human and mouse platelets, the contribution of platelet sema4D/CD100 in both settings is studied. Experiments include 1) complete blood counts, 2) megakaryocyte morphology, 3) fibrinogen binding, 4) α-granule secretion, 5) dense granule secretion, 6) bleeding time, 7) injury-induced carotid artery thrombosis and 8) disseminated thrombosis. Since a platelet phenotype may prove to be subtle (i.e. more like the Akt2 knockout than the Gqα knockout), comparisons are to be made when platelet agonists are added at suboptimal concentrations in the in vitro studies or when prothrombotic stimuli are added at reduced doses in the in vivo studies. Although the sema4D/CD100 mice are backcrossed into the B1/6 strain background for multiple generations, all comparisons are made between littermates produced by Het X Het crosses to avoid strain, age and gender effects.

If platelet sema4D/CD100 contributes to platelet aggregation and thrombosis in mice, then loss of the molecule should protect the mice from thrombotic mortality, prolong the bleeding time and/or result in a failure of complete aggregation or secretion. A complete loss of aggregation or a complete failure to stop bleeding in a bleeding time assay is not expected. Instead, differences between the knockout and the wild type mice are examined that might including the prolonging of the time to occlusion in the carotid artery thrombosis model, partial protection against mortality from disseminated thrombosis, reduced aggregation in response to suboptimal agonist concentrations and premature disaggregation of growing platelet aggregates are examined.

### EXAMPLE 9

### Sema4D/CD100 as a Co-Stimulus for Platelet Activation

Human blood is to be obtained from healthy donors free of all medications. For the aggregation studies, the platelets are studied after isolation from plasma by centrifugation or gel filtration on Sepharose 2B. Whole blood is anticoagulated with the thrombin inhibitor, D-phenyl alanyl-L-prolyl-L-arginine chloromethyl ketone dihydrochloride (PPACK, 93 μM). Platelets are fluorescently labeled with mepacrine and examined at a wall shear rate of 1500 s-1 in a flow chamber coated with type I fibrillar collagen (2.5 mg/ml) and maintained at 37° C. on the stage of a Carl Zeiss Axiovert 135M-LSM410 inverted epifluorescence microscope and laser confocal microscope (Usami, S., et al., Ann Biomed Eng, 1993. 21(1): p. 77-83; Ruggeri, Z. M., J. A. Dent, and E. Saldivar, Blood, 1999. 94: p. 172-178; Savage, B., E. Saldivar, and Z. M. Ruggeri, Cell, 1996. 84(2): p. 289-97). Digital images are analyzed in successive planes, then reconstructed in silico to permit calculation of thrombus volume.

The results with the Sema4D/CD100 knockout mice are most useful when they can be compared with humans. Since there are no reports of humans who harbor sema4D/CD100 mutations or who lack sema4D/CD100 expression, platelet aggregation and the accumulation of platelets on collagen under flow are studied in two sets of conditions: 1) when excess soluble sema4D/CD100 is present to stimulate platelet sema4D/CD100 receptors, and 2) when interactions between sema4D/CD100 and its receptors are blocked.

### EXAMPLE 10

### Examination of Human Platelet Function when the Binding of sema4D/CD100 to its Receptors is Blocked

Preparation of full-length human sema4D/CD100 extracellular domain homodimer is prepared as described above. Monomeric extracellular domain are produced the same way after using site-directed mutagenesis to switch Cys674 to Ala.

Monoclonal Antibodies

Anti-sema4D/CD100 monoclonal antibodies are prepared in mice immunized with recombinant sema4D/CD100 extracellular domain. The antibodies are screened for their ability to block interactions between biotin labeled soluble sema4D/CD100 and HEK-293 cells over-expressing CD72 and plexin-B 1. If a blocking antibody is found, Fab fragments are prepared and used to study the effects of blocking sema4D/CD100 on platelet aggregation and platelet thrombus formation.

Since dimerization of sema4D/CD100 is necessary for its function, the extracellular domain of sema4D/CD100 is generated as a His6-tagged protein in which Cys674 has been mutated to prevent dimerization. The resulting monomer binds but does not activate its receptors (Delaire, S., et al., J Immunol, 2001. 166(7): p. 4348-54). Monoclonal antibodies to sema4D/CD100 are also developed that will block its ability to interact with its receptors. The aggregation and flow chamber studies are then repeated in the presence of the antibodies, using an antibody to αIIbβ3 as a positive control and isotype-matched monoclonal antibodies to irrelevant epitopes as a negative control. The antibodies are generated against recombinant sema4D/CD100 exodomain and screened for their ability to block interactions between biotin-labeled soluble sema4D/CD100 and HEK-293 cells over-expressing CD72 or plexin-B1.

If the binding of sema4D/CD100 to its receptors promotes platelet activation, as the studies with soluble sema4D/CD100 suggest, then blocking those interactions should retard or destabilize the growth of platelet aggregates measured in the aggregometer and flow chamber.

### EXAMPLE 11

### ADAM17 Expression and Activation in Platelets

Human platelets are incubated with PMA and then immunoblotted for ADAM17. This result is obtained with total platelet lysates. First, additional ADAM17 blots are done using physiological platelet agonists and comparing the time course of ADAM17 activation (i.e. appearance of the mature, active form) with the time course of sema4D/CD100 cleavage. Second, the zymogen (100 kDa) and active (75 kDa) forms of ADAM17 on the platelet surface are studied by labeling the platelet surface with a membrane-impermeable biotinylating agent, then isolating biotinylated proteins and immunoblotting for ADAM17. Third, ADAM17 immunoblots are performed on fractionated platelets prepared as described for the sema4D/CD100 localization studies. It is then determined whether ADAM17 is located in α-granules in resting platelets or entirely in the membrane fraction.

### EXAMPLE 12

### Determination of Whether or not ADAM17 is the Actual “Sheddase” for Platelet sema4D/CD100

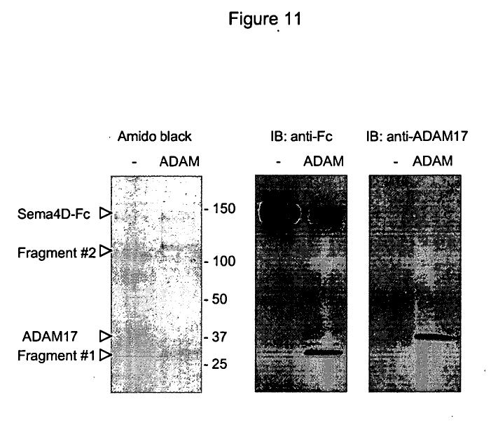

The cleavage site within platelet sema4D/CD100 was determined by using the C-terminal sema4D/CD100 antibody to immunoprecipitate the 25-30 kDa fragment that includes the transmembrane domain and any remaining extracellular domain. FcSema4D, which includes the entire extracellular domain of human sema4D fused via a linker to the N-terminus of the Fc domain of IgG1, was incubated with recombinant ADAM17 lacking the pro domain (Milla, M. E., et al., J Biol Chem, 1999. 274(43): p. 30563-70). Cleavage produced two identifiable fragments (FIG. 11). The smaller of these included the Fc domain. N-terminal sequencing of the Fc-containing fragment from Fc-sema4D yielded SEKTMY/LKQKPKSC where “/” denotes the site of cleavage and the underscored residues are those identified in the sequence analysis. The first two of these residues (LK) are from the sema4D sequence (residues 729 and 730). QKPKSC is from the linker and the start of the IgG1 Fc domain.

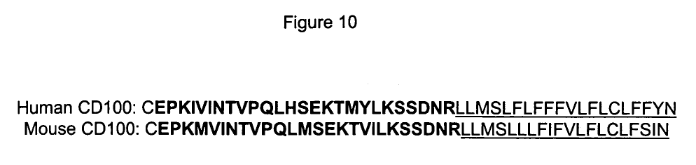

In an effort to determine whether sema4D is a direct substrate for ADAM17, a chimeric protein comprised of the extracellular domain of human sema4D fused to the N-terminus of an IgG Fc domain fragment (Ishida et al. 2003. Int Immunol 15:1027-1034) was incubated with recombinant ADAM17 lacking the pro domain (Milla et al. 1999. J Biol Chem 274:30563-30570). Cleavage produced two identifiable fragments. N-terminal sequencing of the smaller, Fc-containing fragment identified a cleavage site within Fc-sema4D corresponding to sema4D residues Y728 and L729, just external to the predicted transmembrane domain (Hall et. al. 1996. Proc Natl Acad Sci U S A 93:11780-11785). It is also not conserved between human and mouse sema4D: the human MY/LK sequence is VI/LK in mouse (FIG. 10). FIG. 10 shows the sequence spanning the region of sema4D/CD100 in which cleavage may occur. The boundaries are set by the transmembrane domain and the cysteine residue needed for the homodimerization of sema4D/CD100. It has been determined that in platelets, as in lymphocytes, the extracellular domain of sema4D/CD100 is shed as a disulfide-linked dimer. Although some proteases cleave surface proteins in the transmembrane domain, ADAM17 does not.

### EXAMPLE 13

### Cleavage of sema4D/CD100 from the Platelet Surface

A comparison is made of platelet aggregation and injury-induced thrombus formation in irradiated mice reconstituted with fetal liver from 1) sema4D/CD100(−/−) mice, 2) wild type mice and 3) TACE(ΔZn/ΔZn) mice. This yields platelets that have either no sema4D/CD100, normal sema4D/CD100 or non-sheddable sema4D/CD100. There are a number of caveats to this experiment. One is that it is done with irradiated mice. A second is that the “non-shedding” TACE(ΔZn/ΔZn)-reconstituted mice also lack the ability to shed proteins other than sema4D/CD100, such as GP Ibα Bergmeier, W., et al., Circ Res, 2004. 95(7): p. 677-683).

If surface-associated sema4D/CD100 is enough for platelet activation, then the TACE(ΔZn/ΔZn) chimeric platelets may function normally and behave like the wild type mice in the aggregometer and thrombosis assays. If platelet sema4D/CD100 has to be shed to be functional, then the TACE(ΔZn/ΔZn) chimeras should behave like the sema4D/CD100 knockout mice.

### EXAMPLE 14

### Expression and Localization of CD72 in Human Platelets

It is to be determined where CD72 is located within resting platelets, the kinetics of the appearance of CD72 on the platelet surface in response to agonists are measured and the splice variants of CD72 that are expressed in human platelets are identified. The increase in CD72 surface expression seen in the FACS studies suggests that at least some of the CD72 in resting platelets is initially intracellular, either in the surface connecting system or the α-granule membranes. Using fluorescence microscopy, the staining patterns of antibodies to CD72, αIIbβ3 and von Willebrand factor (VWF) was compared on platelets that had been allowed to spread on fibrinogen. VWF served as a marker for platelet α-granules. αIIbβ3 served as a marker for the platelet surface, as well as intracellular membranes. The distribution of CD72 resembled that of αIIbβ3 (but with less intensity owing to the large difference in expression levels) and was distinctly different from that of VWF (data not shown). This result suggests that CD72 is unlikely to be in α-granule membranes. CD72 expression may precede the loss of sema4D/CD100 so that CD72 is available to interact with sema4D/CD100 before and after the sema4D/CD100 is shed.

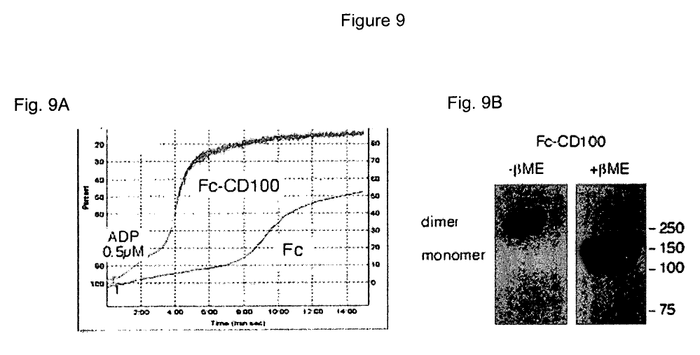

Data thus far suggest that platelets are a source of sema4D/CD100, but also capable of responding to it. Soluble sema4D/CD100 caused an increase in ADP-induced aggregation (FIG. 9). In this experiment, soluble sema4D/CD100 was added as a fusion protein (referred to as FcSema4D or FcCD100) in which the entire extracellular domain of sema4D/CD100 was fused to an IgG Fc domain. The protein forms a disulfide-linked homodimer capable of activating sema4D/CD100 receptors (Ishida, I., et al., Int Immunol, 2003. 15(8): p. 1027-34). FcCD100 had no effect when added on its own (not shown). However, when added in the presence of ADP, it caused an increase in platelet aggregation. Recombinant Fc did not. Therefore, it appears that stimulating a receptor for sema4D/CD100 on platelets potentiates platelet responses to ADP. Additional platelet agonists (especially collagen) are going to be explored using platelet accumulation on collagen under flow as an additional endpoint.

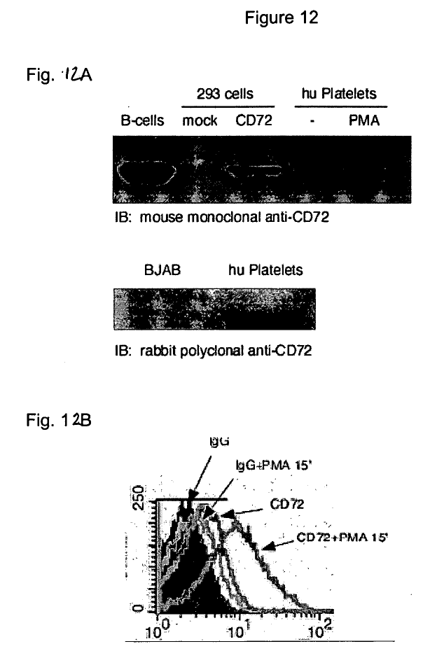

At present there are only two known receptors for sema4D/CD100 : CD72 and plexin-B1. These studies consider whether either (or both) of them plays a role in platelet responses to sema4D/CD100. CD72 is a type II transmembrane protein that is thought to function as a homodimer. There are no prior reports of CD72 being expressed on platelets, but it was detected in human platelet lysates with two different antibodies (FIG. 12). Using a third antibody which is directed against the extracellular domain of CD72, it was also detected by FACS. There appears to be only a modest amount on the surface of resting platelets, but this level increases approximately 3-fold (2.9±0.1, mean±SEM, N=3) when platelets are activated. Once on the surface, CD72 expression remains stable for at least 60 min at 37° C.

### EXAMPLE 15

### Signaling by CD72 in Platelets

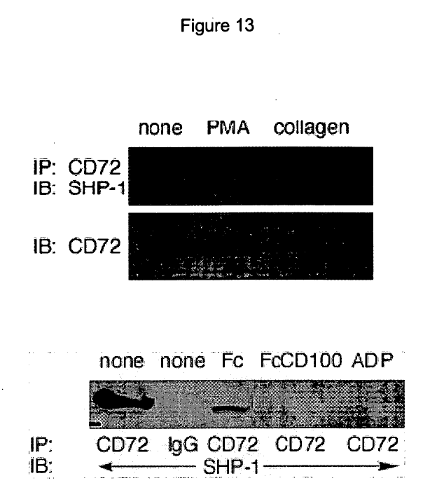

The best-described mechanism for signaling downstream of CD72 involves the tyrosine phosphatase SHP-1 (Wu, Y., et al., Curr Biol, 1998. 8(18): p. 1009-17; Adachi, T., et al., J Immunol, 1998. 160(10): p. 4662-5. Hokazono, Y., et al., J Immunol, 2003. 171(4): p. 1835-43). It is known that platelets express SHP-1 (Li, R. Y., et al., Cell Mol Biol (Noisy-le-grand), 1994. 40(5): p. 665-75; Li, R. Y., et al., EMBO J., 1995. 14: p. 2519-2526; Jones, M. L. and A. W. Poole, Methods Mol Biol, 2004. 273: p. 169-78.). Since stimulating human platelets with soluble FcCD100 potentiates platelet activation, it is most likely that SHP-1 is associated with CD72 in resting platelets, where it serve's as a brake on unwarranted platelet activation, and that platelet activation releases this brake by causing the dephosphorylation of CD72, releasing the SH2 domain of SHP-1. This would not only inactivate the phosphatase, but also dissociate it from the membrane. As shown in FIG. 13, human platelets were incubated with an agonist or with FcCD100 or Fc, then lysed and immunoprecipitated with rabbit anti-CD72 (or rabbit IgG) before being immunoblotted with monoclonal anti-SHP-1 or anti-CD72 as indicated. The results show that SHP-1 co-precipitates with CD72 in resting platelets, but not in activated platelets or platelets incubated with FcCD100.

### EXAMPLE 16

### Characterization of the CD72 Knockout Mice

The same studies are performed as were used to characterize the sema4D (−/−) mice. All comparisons will be made between littermates produced by crossing heterozygous CD72(±) mice. Soluble sema4D/CD100 is added to determine whether the potentiating effect observed that we observed is mediated by CD72 . The carotid artery injury assay will be performed to see if there are differences in vivo.

CD72 knockout mice have been obtained and have been identified by PCR genotyping. The absence of CD72 should reduce SHP-1 activity in platelets and make them more responsive to agonists which would be consistent with the enhanced responsiveness to ADP that was observed when human platelets were incubated with soluble sema4D/CD100.

### EXAMPLE 17

### Expression of plexin-B1 in Platelets

Plexin-B1 is an interesting molecule in the context of platelets for a number of reasons. Not only is it a high affinity receptor for a molecule that expressed and shed by platelets, but it also presents the potential for a variety of signaling mechanisms that could be relevant to the late events of platelet activation, including effects on Rho and Rac that might help to maintain the active state of the platelet cytoskeleton (Aurandt, J., et al., LARG. Proc Natl Acad Sci USA, 2002. 99(19): p. 12085-90; Perrot, V., J. Vazquez-Prado, and J. S. Gutkind, J Biol Chem, 2002. 277(45): p. 43115-20; Swiercz, J. M., et al., Neuron, 2002. 35(1): p. 51-63; Oinuma, I., et al., J Biol Chem, 2003. 278(28): p. 25671-7; Oinuma, I., et al., Science, 2004. 305(5685): p. 862-5) and tyrosine kinase based signaling if plexin-B1 associates with receptor tyrosine kinases in platelets as it does in other types of cells (Giordano, S., et al., Nat Cell Biol, 2002. 4(9): p. 720-4.).

In situ hybridization studies of adult and embryonic mouse tissues are performed to see where plexin-B1 is expressed. As part of that screen megakaryocytes located in the spleen are also examined to determine if the express plexin-B1 mRNA. The in situ hybridization is performed using a 700 nt 3′ untranslated region probe that was amplified from mouse brain. RT-PCR is also used to to look directly for plexin-B1 mRNA in platelets, using appropriate controls to exclude contamination by leukocytes as described earlier.

### EXAMPLE 18

### The Response of Monocytes and Endothelial Cells to Platelet-Derived sema4D/CD100

The studies on tissue factor expression are performed with normal human monocytes and with the monocytic THP-1 cell line. THP-1 cells are available from the ATCC. Human monocytes are isolated by negative selection from peripheral blood mononuclear cells as described, (Delaire, S., et al., J Immunol, 2001. 166(7): p. 4348-54), using density gradient centrifugation (Lymphoprep, Axis-Shield, Oslo) and a monocyte isolation kit (MACS, Miltenyi Biotec, Germany). Changes in factor mRNA and protein expression over time when monocytes are incubated with Fc-CD100 or recombinant sema4D/CD100 exodomain are detected by quantitative real-time PCR and flow cytometry using antibody TF9-9C3 from American Diagnostica (Lindmark, E., T. Tenno, and A. Siegbahn, Arterioscler Thromb Vasc Biol, 2000. 20(10): p. 2322-8.) Thrombin serves as a positive control, but it is also determined whether soluble sema4D/CD100 potentiates the effects of sub-optimal concentrations of thrombin in addition to or instead of stimulating TF expression by itself.

Human umbilical vein endothelial cells (HUVEC) are obtained (Molino, M., et al., G. J. Biol. Chem., 1995. 270: p. 11168-11175; Molino, M., et al., J. Biol. Chem., 1997. 272: p. 11133-11141; O'Brien, P. J., et al., J. Biol. Chem., 2000. 275: p. 13502-13509). Tissue factor expression by HUVEC is incubated with soluble sema4D/CD100, thrombin, anti-CD40 and TNFα (alone and in combination) is measured by western blotting and by quantitative PCR. Expression of E-selectin, VCAM-1 and ICAM-1 is measured by flow cytometry. Migration assays are also performed (Wagner, A. H., et al., Arterioscler Thromb Vasc Biol, 2004. 24(4): p. 715-20.) in settings with acute thrombosis or in diseases such as lupus or some solid tumors.

Soluble dimeric sema4D/CD100 has been shown to inhibit the spontaneous and chemokine-induced migration of monocytes (Delaire, S., et al., J Immunol, 2001. 166(7): p. 4348-54) and to stimulate formation and release of pro-inflammatory cytokines from monocytes (Ishida, I., et al., Int Immunol, 2003. 15(8): p. 1027-34). The sema4D/CD100 receptor on monocytes has not been identified. There is conflicting information about CD72: anti-CD72 antibodies can mimic the effects of soluble sema4D/CD100 (Ishida, I., et al., Int Immunol, 2003. 15(8): p. 1027-34), but at least one review states that CD72 could not be detected (Bismuth, G. and L. Boumsell, Sci STKE, 2002. 2002(128): p. RE4).

Since the reported effects of soluble sema4D/CD100 on monocytes are presumably unrelated to the origin of the soluble sema4D/CD100, it is reasonable to expect that platelet-derived sema4D/CD100 would produce the same responses. Moreover, it is also likely that more sema4D/CD100 is shed from platelets than from lymphocytes in disorders marked by local thrombosis or widespread platelet activation. It is probable that platelet-derived sema4D/CD100 inhibits the further migration of monocytes that have been attracted to sites of vascular disease or injury by chemokine release and that, once in the area, monocytes contribute to local damage in at least two ways: through the release of inflammatory cytokines and by up-regulation of tissue factor expression on monocytes.

As with monocytes, there is already evidence that endothelial cells can respond to soluble sema4D/CD100, although, these experiments were not performed with platelets as the source of the sema4D/CD100. Basile et al., have recently shown that plexin-B1 is expressed in human umbilical vein (HUVEC) and porcine aorta endothelial cells (Basile, J. R., et al., Cancer Res, 2004. 64(15): p. 5212-24). Soluble sema4D/CD100 is a chemoattractant for both types of endothelial cells and also caused tube formation. These effects did not appear to involve Met, but did depend on an intact C-terminus, suggesting that an interaction with a PDZ domain-containing cytosolic protein was required. These results raise the possibility that sema4D/CD100 shed from the surface of platelets at a site of thrombosis can affect endothelial cell behavior, either by itself or in concert with other platelet-derived products. This may prove to be relevant where chronic activation of platelets may provide an ongoing source of soluble sema4D/CD100 in the circulation.

It is to be determined whether soluble sema4D/CD100 has a prothrombotic or pro-inflammatory effect on endothelial cells. CD40L provides a useful paradigm for these studies since it has been shown to have both types of effects, causing an increase in tissue factor expression and an increase in surface molecules that play a role in the migration of inflammatory cells (Henn, V., et al., Nature, 1998. 391: p. 591-594; Slupsky, J. R., et al., Thromb. Haemost., 1998. 80: p. 1008-1014.). Since monocytes also have receptors for sema4D/CD100, transmigration assays are performed to look at the movement of THP-1 cells through a HUVEC monolayer.

### EXAMPLE 19

### Assay for Soluble sema4D/CD100 in Human Plasma

The antibodies to sema4D/CD100 are made by immunizing Balb/c mice with recombinant sema4D/CD100 exodomain prepared as described above. Following a standard protocol for boosting the mice with two repeated injections, serum samples from each mouse are tested by ELISA with the recombinant protein and the mouse with the highest titer are sacrificed and its spleen cells used to make hybridomas. The hybridoma supemates are screened by ELISA and by FACS using resting platelets and Jurkat T-cells. Positive wells are identified in this manner and are subjected to cloning by dilution and productive clones will be isolated. An appropriate pair of antibodies that can be used in an ELISA assay are identified empirically.

The results show that platelet activation can cause all of the sema4D/CD100 present in platelets to be cleaved and shed. This process is completed in vitro within 30 minutes. The number of copies of sema4D/CD100 per platelet is not known, but using a conservative estimate of 500-1,000 copies per platelet, a platelet concentration of 3×10⁸ platelets/ml and a mass of 120 kDa for the exodomain fragment, yields a final concentration 30-60 ng/ml (0.25-0.5 nM). Local concentrations near a platelet thrombus might be higher, and the concentration in the gaps between aggregated platelets may prove to be higher yet. For comparison, the effects of soluble sema4D/CD100 on monocytes are half-maximal at 5-10 ng/ml and maximal at 40-50 ng/ml (Delaire, S., et al., J Immunol, 2001. 166(7): p. 4348-54).

An assay is to be developed that can be used to detect soluble sema4D/CD100 in plasma and it is used to determine levels in normal individuals. This assay is also used to measure soluble sema4D/CD100 levels in three groups of individuals who are predicted to have elevated levels because of platelet activation occurring in vivo: (1) patients undergoing cardiopulmonary bypass (CBP); (2) patients with heparin-induced thrombocytopenia (HIT); and (3) patients with atherosclerotic cardiovascular disease. The assay of the present invention is a classical ELISA using two monoclonal antibodies with non-overlapping epitopes in the extracellular domain of sema4D/CD100. The first antibody is attached to a 96 well microtiter plate and used to capture soluble sema4D/CD100 from diluted plasma samples. The second antibody is biotinylated and used to detect the captured soluble sema4D/CD100 following the addition of horseradish peroxidase (HRP)-conjugated avidin. Standard curves are generated using recombinant sema4D/CD100 exodomain.

Once an assay has been established that works with recombinant sema4D/CD100 and with the supernate of platelets activated in vitro, a normal range is determined using healthy adult volunteers 20-30 years of age, 20 male and 20 female. It is expected that little, if any, soluble sema4D/CD100 is present in plasma in normal conditions.

### EXAMPLE 20

### Changes in Soluble sema4D/CD100 Levels During Cardiopulmonary Bypass

Inclusion Criteria:

Patients included in this study are those who had cardiac catheterization and are to undergo cardiopulmonary bypass for cardiac valve disease.

Exclusion Criteria:

A subject is excluded if he/she: (1) has a baseline platelet count <150,000/mm3; (2) is on chronic heparin therapy; (3) has a history of thrombosis or a known risk factor for thrombosis, such as malignancy or a myeloproliferative disorder; (4) has a current or past diagnosis of HIT.

Data Collection

Enrolled patients have baseline blood and urine samples drawn on Day 0, i.e. prior to beginning heparin for CPB and daily for at least 5 days, and for 7 days if the patient remains hospitalized, after CPB and again on Day 30. Patients undergoing cardiopulmonary bypass should provide a good positive control for the soluble sema4D/CD100 assay. There is already an extensive literature on platelet activation and the release of α-granule proteins and CD40L during bypass (Komai, H. and S. G. Haworth, Ann Thorac Surg, 1994. 58(2): p. 478-82; Cella, G., et al., Eur J Clin Invest, 1981. 11(3): p. 165-9; Nannizzi-Alaimo, L., et al., Circulation, 2002. 105: p. 2849-2854.). These patients also have a transient drop in platelet count and biochemical evidence of platelet activation, including an increase in TxB2 levels (Kobinia, G. S., et al., J Thorac Cardiovasc Surg, 1986. 91(6): p. 852-7). The patients that are enrolled are individuals scheduled for cardiac valve replacement or repair procedures that require cardiopulmonary bypass with exposure to heparin. Samples are taken prior to surgery, during surgery and on a daily basis post-operatively.

Platelet activation in the bypass circuit should lead to the shedding of sema4D/CD100, which is detectable. Over time, levels of soluble sema4D/CD100 in an otherwise uncomplicated patient should fall, providing an opportunity to test samples from an initial baseline normal level, to an increased level during surgery, followed by a fall during recovery.

### EXAMPLE 21

### Changes in Soluble sema4D/CD100 Levels when Patients Develop Heparin-Induced Thrombocytopenia

Subjects are obtained as described above. Heparin-induced thrombocytopenia (HIT) typically occurs when individuals receive heparin in a setting that includes platelet activation. Activated platelets release PF4 from their α-granules. PF4 binds heparin and the resulting complex can be antigenic, giving rise to antibodies that can bind to platelets and cause platelet activation in the circulation, which in turn can cause thrombocytopenia and, in some cases, arterial and venous thrombosis. The platelet activation that occurs in patients with HIT should lead to the cleavage of sema4D/CD100 and an increase in plasma sema4D/CD100 levels. It may also be that platelet-derived sema4D/CD100 contributes to the development of this disorder through effects on antibody development.

### EXAMPLE 22

### Circulating sema4D/CD100 Levels in Patients with Atherosclerotic Cardiovascular Disease

Subjects are patients undergoing coronary angiography. If the subject gave written informed consent, 20 ml of blood was drawn at the time of catheter insertion. The fasting sample was transferred to one 10 ml EDTA tube and one serum separator tube. Triglycerides, total cholesterol and HDL-cholesterol were assayed on all samples. Additional plasma and serum samples have been stored a −80° C. since their collection. Additionally, DNA has been isolated from leukocytes. Data including reason for procedure, past cardiovascular events, cardiac risk factors (diabetes, smoking, and hypertension), medications, and angiography results are collected from the subjects' clinical catheterization report and medical record and entered into a study database that includes no personal identifiers. Between July 1998 and March 2003, 3850 subjects were enrolled. From this large group, a subset of cases and controls has been identified. Subjects undergoing catheterization for cardiomyopathy, pre-operative or anniversary catheterization were excluded. Group 1, the control group, consists of patients that were evaluated for coronary artery disease (CAD) but found to have no evidence of atherosclerotic disease as cause of symptoms. Individuals in Group 2, the “non-emergent” CAD group, have 70% or greater stenosis in the left main coronary artery alone or in two or more of the left anterior descending (LAD), the right coronary artery (RCA) or the circumflex artery, but have not had a myocardial infarction. Group 3 includes “emergent patients” experiencing an acute MI and who had a stenosis >20%. Group 1, 2 and 3 cases were matched by age and gender. The females identified for this subset include a total of 150 controls, 150 cases and 58 acute cases. The males identified for this subset include a total of 150 controls, 150 cases and 149 acute cases. A selection of matched individuals in the three groups is performed followed by a measurement of soluble sema4D/CD100 and analysis of the results.

Statistical Analysis:

For clinical data, variables will be compared using the Chi2 test. ANOVA will be used to compare the three groups. Statistical significance will be indicated by a p<0.05.

There is considerable evidence that platelets are active participants in the development of atherosclerotic cardiovascular disease, as well as contributors to the acute thrombotic events that can be triggered by plaque rupture (Naghavi, M., et al., Circulation, 2003. 108(15): p. 1772-8; Naghavi, M., et al., Circulation, 2003. 108(14): p. 1664-72. Huo, Y. Q., et al. Nat. Med., 2003. 9: p. 61-67. Huo, Y. and K. F. Ley, Trends Cardiovasc Med, 2004. 14(1): p. 18-22. Massberg, S., C. Schulz, and M. Gawaz, Semin Vasc Med, 2003. 3(2): p. 147-62. Aukrust, P., et al., Circulation, 1999. 100: p. 614-620). That includes the recent demonstration by members of this SCCOR that PF4 can be found in the vessel wall near sites of plaque formation (Pitsilos, S., et al., Thromb Haemost, 2003. 90(6): p. 1112-20; Sachais, B. S., et al., Semin Thromb Hemost, 2004. 30(3): p. 351-8) Patients with atherosclerotic cardiovascular disease are predicted to have a more subtle degree of ongoing platelet activation and less of an increase in soluble sema4D/CD100 levels than the cardiopulmonary bypass and HIT patients.

In this case, a database of samples is used that has already been collected and stored from patients. This is a group of nearly 4,000 patients from whom extensive clinical and laboratory information was collected prior to their undergoing cardiac angiography. It is likely that atherosclerotic cardiovascular disease is associated with ongoing platelet activation that results in the shedding of sema4D/CD100 from the platelet surface and that patients with greater extents of disease will have higher sema4D/CD100 levels than those with lesser extents of disease.

Each and every patent, patent application and publication that is cited in the foregoing specification is herein incorporated by reference in its entirety.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims.

What is claimed is:

1. A method of treating at least one symptom of a platelet disorder or an endothelial cell disorder in a human, said method comprising administering a sema4D/CD100 modulator to said human.

2. The method of claim 1, wherein said platelet disorder is selected from the group consisting of heart attack, stroke, cardiopulmonary bypass disease, heparin-induced thrombocytopenia, atherosclerosis, thrombosis, thrombotic microangiopathy, disseminated intravascular coagulation, acquired platelet disorder and inherited platelet disorder.

3. The method of claim 1, wherein said endothelial cell disorder is selected from the group consisting of thrombosis, arteriosclerosis and aneurysm.

4. The method of claim 1, wherein said endothelial cell disorder comprises angiogenesis.

5. The method of claim 4, wherein said angiogenesis occurs during the metastasis of a cancer.

6. The method of claim 1, wherein said treating step comprises reducing platelet aggregation.

7. The method of claim 4, wherein said platelet aggregation comprises collagen-induced platelet aggregation or thrombin-induced aggregation.

8. The method of claim 1, wherein said modulator consists essential of an inhibitor of sema4D/CD100.

9. The method of claim 8, wherein said inhibitor prevents or inhibits sema4D/CD100 from binding to its receptor.

10. The method of claim 9, wherein said receptor consists essentially of CD72 or plexin B1.

11. The method of claim 8, wherein said inhibitor prevents or inhibits cleavage of sema4D/CD100.

12. The method of claim 9, wherein said cleavage of sema4D/CD100 is achieved by activity of a metalloprotease.

13. A method for identifying a test compound which modulates sema4D/CD100 activity, said method comprising:
(a) contacting a cell expressing sema4D/CD100 with said test compound;
(b) measuring an activity of sema4D/CD100 in the presence of the test compound and in the absence of the test compound; and
(c) comparing said activity of sema4D/CD100 in the presence of said test compound with said activity of sema4D/CD100 in the absence of said test compound, thereby determining sema4D/CD100 activity of the test compound.

14. The method of claim 13, wherein said cell expressing sema4D/CD100 is selected from the group comprising a platelet, an endothelial cell and a monocyte.

15. The method of claim 13, wherein said modulator of sema4D/CD100 activity increases or decreases cleavage of sema4D/CD100.

16. The method of claim 15, wherein said cleavage is generated by cleavage with a metalloproteinase.

17. The method according to claim 13, wherein binding of said test compound to sema4D/CD100 modulates sema4D/CD100 activity.

18. The method according to claim 11, wherein binding of said test compound to a sema4D/CD100 receptor modulates sema4D/CD100 activity.

19. The method of claim 18, wherein said sema4D/CD100 is CD72 or plexin B1.

20. The method according to claim 13, wherein the cell is selected from an established mammalian cell line.

21. A method of screening for a compound having anti-thrombotic or anti-platelet activity comprising:
(a) contacting platelets in vitro with a test compound; and
(b) monitoring activity of said compound to inhibit platelet aggregation through inhibition of sema4D/CD100 cleavage, wherein activity as an inhibitor of sema4D/CD100 binding is indicative of anti-thrombotic or anti-platelet activity of said compound.

22. The method of claim 21, wherein said platelet aggregation is selected from the group consisting of collagen-induced aggregation, heparin-induced and thrombin-induced platelet aggregation.

23. A method of diagnosing a platelet disorder and/or endothelial cell disorder or similar condition in a mammal, said method comprising:
(a) obtaining a biological sample from said mammal;
(b) determining a presence or level of sema4D/CD100 in said biological sample;
(c) comparing the level of sema4D/CD100 in said biological sample with the level of sema4D/CD100 in a biological sample obtained from a like mammal not afflicted with a platelet disorder and/or endothelial cell disorder, wherein a higher level of sema4D/CD100 in said biological sample from said mammal compared with the level of sema4D/CD100 in said biological sample from said like mammal is an indication that said mammal is afflicted with a platelet disorder and/or endothelial cell disorder, thereby diagnosing the platelet disorder and/or endothelial cell disorder in said previously undiagnosed mammal.

24. A kit for treating at least one symptom of a platelet disorder or an endothelial cell disorder in a human in accordance with claim 1.

25. A kit for identifying a test compound which modulates sema4D/CD100 activity in accordance with claim 13.

26. A kit for diagnosing a platelet disorder and/or endothelial cell disorder or similar condition in a mammal in accordance with claim 23.


SEQUENCE LISTING (2 sequences)

SEQ ID NO: 1 - protein, 47 aa, Homo sapiens
CEPKIVINTVPQLHSEKTMYLKSSDNRLLMSLFLFFFVLFLCLFFYN

SEQ ID NO: 2 - protein, 47 aa, Homo sapiens
CEPKMVINTVPQLMSEKTVILKSSDNRLLMSLLLFIFVLFLCLFSIN